(19) United States
(12) Patent Application Publication
(10) Pub. No.: US 2025/0037876 A1
(43) Pub. Date: Jan. 30, 2025

(54) SYSTEMS AND METHODS FOR DEVELOPING AND UTILIZING A HEMATOLOGIC PROGNOSTIC CLASSIFIER

(71) Applicant: GRAIL, LLC, Menlo Park, CA (US)

(72) Inventors: Yuefan HUANG, Houston, TX (US); Alvin SHI, Palo Alto, CA (US); Qinwen LIU, Fremont, CA (US); Oliver Claude VENN, San Francisco, CA (US); Rita SHAKNOVICH, Menlo Park, CA (US)

(73) Assignee: GRAIL, LLC, Menlo Park, CA (US)

(21) Appl. No.: 18/785,786

(22) Filed: Jul. 26, 2024

Related U.S. Application Data
(60) Provisional application No. 63/515,911, filed on Jul. 27, 2023.
(60) Provisional application No. 63/598,529, filed on Nov. 13, 2023.

(51) Int. Cl. G16H 50/30; G16B 30/00; G16H 10/40; G16H 50/20

(52) U.S. Cl. CPC G16H 50/30; G16B 30/00; G16H 10/40; G16H 50/20

(57) ABSTRACT

Systems and methods of the disclosure may include a computer-implemented method, the computer-implemented method including: receiving, at a computer system, nucleic acid sequencing data derived from a methylation assay performed on a biological sample associated with at least one subject; computing, using a processor associated with the computer system, a beta value matrix based on the nucleic acid sequencing data, wherein the beta value matrix comprises one or more missing beta values; addressing, using the processor, the one or more missing beta values in the beta value matrix using a missing beta value completion approach; identifying, using the processor, one or more principal components in the completed beta value matrix; and training, using the one or more principal components in combination with a predetermined set of clinical variables, a classifier to predict a survival outcome for a target subject associated with a disease type.

### CROSS REFERENCE TO RELATED APPLICATIONS

The application claims priority to U.S. Provisional Application No. 63/515,911, filed on Jul. 27, 2023, and U.S. Provisional Application No. 63/598,529, filed on Nov. 13, 2023, which are both incorporated by reference herein in their entireties.

### TECHNICAL FIELD

The present disclosure relates generally to prognostic tools for hematologic malignancies and, more specifically, to systems and methods for developing and employing a prognostic classifier for survival outcome prediction in blood cancers.

### BACKGROUND

Hematologic malignancies, or blood cancers, present significant challenges in prognosis due to their biological complexity and heterogeneity. Current risk stratification methods, which rely on cytogenetics, mutations, and clinical parameters, are limited to single hematologic indications. Accordingly, there is a need for more comprehensive prognostic tools that can address the diverse nature of blood cancers and provide accurate risk assessments using minimally invasive methods.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, systems and methods are described for developing and utilizing a pan-heme prognostic classifier for survival outcome prediction in blood cancers.

In one aspect, a computer-implemented method is disclosed. The computer-implemented may include: receiving, at a computer system, nucleic acid sequencing data derived from a methylation assay performed on a biological sample associated with at least one subject; computing, using a processor associated with the computer system, a beta value matrix based on the nucleic acid sequencing data, wherein the beta value matrix comprises one or more missing beta values; addressing, using the processor, the one or more missing beta values in the beta value matrix using a missing beta value completion approach; identifying, using the processor, one or more principal components in the completed beta value matrix; and training, using the one or more principal components in combination with a predetermined set of clinical variables, a classifier to predict a survival outcome for a target subject associated with a disease type.

In another aspect, a system is disclosed. The system may include: one or more processors; one or more computer readable media storing instructions that are executable by the one or more processors to perform operations to: receive nucleic acid sequencing data derived from a methylation assay performed on a biological sample associated with at least one subject; compute a beta value matrix based on the nucleic acid sequencing data, wherein the beta value matrix comprises one or more missing beta values; address the one or more missing beta values in the beta value matrix using a missing beta value completion approach; identify one or more principal components in the completed beta value matrix; and train, using the one or more principal components in combination with a predetermined set of clinical variables, a classifier to predict a survival outcome for a target subject associated with a disease type.

In yet another aspect, a non-transitory computer-readable medium is disclosed. The non-transitory computer-readable medium may store computer-executable instructions which, when executed by a system, cause the system to perform operations including: receiving, at a computer system, nucleic acid sequencing data derived from a methylation assay performed on a biological sample associated with at least one subject; computing, using a processor associated with the computer system, a beta value matrix based on the nucleic acid sequencing data, wherein the beta value matrix comprises one or more missing beta values; addressing, using the processor, the one or more missing beta values in the beta value matrix using a missing beta value completion approach; identifying, using the processor, one or more principal components in the completed beta value matrix; and training, using the one or more principal components in combination with a predetermined set of clinical variables, a classifier to predict a survival outcome for a target subject associated with a disease type.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

The accompanying drawings, which are incorporated in and constitute a part of this specification, illustrate several embodiments and together with the description, serve to explain the principles of the disclosure.

FIG. 1A depicts an exemplary computer system for executing the methods described herein, according to one or more embodiments of the present disclosure.

FIG. 1B depicts an exemplary software platform for executing the methods described herein.

FIG. 2A depicts an exemplary workflow for a method for providing a prognostic classifier designed to predict survival outcomes for subjects with various hematologic malignancies, according to one or more embodiments of the present disclosure.

FIG. 2B depicts another exemplary workflow of a method for constructing a prognostic model using methylation data to predict survival outcomes in subjects with various hematologic malignancies, according to one or more embodiments of the present disclosure.

FIG. 3 depicts a diagram that presents an overview of the population characteristics used in the study to develop the prognostic classifier, according to one or more embodiments of the present disclosure.

FIG. 4 depicts a diagram providing a visual comparison of the performance of different trained models to predict survival outcomes in subjects with hematologic malignancies, according to one or more embodiments of the present disclosure.

FIGS. 5A-5F depict variable importance plots for the baseline model across six nested cross-validation folds, according to one or more embodiments of the present disclosure.

FIGS. 6A-6F depict variable importance plots for the clinical methyl median model across six nested cross-validation folds, according to one or more embodiments of the present disclosure.

FIG. 7 depicts a Kaplan-Meier plot associated with pan-heme risk classification, according to one or more embodiments of the present disclosure.

FIG. 8 depicts a Kaplan-Meier plot associated with chronic lymphocytic leukemia small lymphocytic lymphoma (CLL SLL), according to one or more embodiments of the present disclosure.

FIG. 9 depicts a Kaplan-Meier plot associated with follicular lymphoma, according to one or more embodiments of the present disclosure.

FIG. 10 depicts a Kaplan-Meier plot associated with polycythemia vera, according to one or more embodiments of the present disclosure.

FIG. 11 depicts a Kaplan-Meier plot associated with plasma cell myeloma, according to one or more embodiments of the present disclosure.

FIG. 12 depicts a Kaplan-Meier plot associated with Hodgkin Lymphoma, according to one or more embodiments of the present disclosure.

FIG. 13 depicts a Kaplan-Meier plot associated with monoclonal gammopathy of undetermined significance (MGUS), according to one or more embodiments of the present disclosure.

FIG. 14 depicts a Kaplan-Meier plot associated with diffuse large B-cell lymphoma (DLBCL), according to one or more embodiments of the present disclosure.

FIG. 15 depicts a Kaplan-Meier plot associated with mucosa-associated lymphoid tissue (MALT) nodal marginal zone lymphoma (NMZL), according to one or more embodiments of the present disclosure.

FIG. 16 depicts a Kaplan-Meier plot associated with mantle cell lymphoma, according to one or more embodiments of the present disclosure.

FIG. 17 depicts a Kaplan-Meier plot associated with B-cell lymphoma, according to one or more embodiments of the present disclosure.

FIG. 18 depicts a Kaplan-Meier plot associated with myelodysplastic syndrome (MDS), according to one or more embodiments of the present disclosure.

FIG. 19 depicts a volcano plot that illustrates significant differentially methylated regions (DMRs) identified between high-risk and low-risk participant groups, according to one or more embodiments of the present disclosure.

FIGS. 20A and 20B depict histograms that collectively present the results of DMR analysis between high-risk and low-risk groups, according to one or more embodiments of the present disclosure.

FIG. 21 depicts a bar chart that illustrates the total length of significant DMRs in various genic features, according to one or more embodiments of the present disclosure.

FIG. 22 depicts a bar chart that illustrates the total length of significant DMRs in various CpG-related features, according to one or more embodiments of the present disclosure.

FIGS. 23A and 23B depict bar charts that compare the distribution of significant DMRs to the background in terms of genic and CpG-related features, according to one or more embodiments of the present disclosure.

FIGS. 24A-24D depict histograms that present the delta beta values for significant DMRs related to CpG features, according to one or more embodiments of the present disclosure.

FIGS. 25A-25G depict histograms that present the delta beta values for significant DMRs related to CpG features, according to one or more embodiments of the present disclosure.

FIGS. 26A and 26B depict panels that collectively illustrate KEGG enrichment analysis for DMRs in high-risk versus low-risk groups, according to one or more embodiments of the present disclosure.

FIG. 27 depicts an exemplary workflow for a method for constructing a prognostic classifier designed to predict survival outcomes for subjects with various hematologic malignancies, according to one or more embodiments of the present disclosure.

FIG. 28 depicts an example computing system, according to one or more embodiments of the present disclosure.

### DETAILED DESCRIPTION OF EMBODIMENTS

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

Hematologic malignancies, commonly known as blood cancers, encompass a wide range of diseases including leukemia, lymphoma, and myeloma. These malignancies originate in the blood-forming tissues, such as bone marrow and the lymphatic system, leading to uncontrolled abnormal blood cell growth that disrupts normal blood cell function. Prognosis in these malignancies is particularly challenging due to the complex and diverse biology of the diseases, coupled with the difficulties associated with invasive diagnostic procedures like bone marrow aspiration. Accurate risk stratification is important for effective treatment planning and improving subject outcomes. More particularly, accurate risk stratification enables clinicians to categorize subjects based on their likelihood of disease progression and overall survival. By identifying high-risk and low-risk subjects, healthcare providers may tailor treatment plans to individual needs. For instance, high-risk subjects may require more aggressive and immediate treatments, or may be willing to try more experimental treatments, whereas low-risk subjects may benefit from less intensive therapies. Additionally, stratifying subjects accurately may help in more reliably predicting the disease course, which may enable clinicians to provide subjects with a clearer understanding of their expected outcomes.

Current prognostic tools are often limited to single hematologic indications and fail to address the biological heterogeneity across the different types of blood cancers. More particularly, conventional prognostic methods for addressing hematologic malignancies include cytogenetic, mutational, and/or clinical parameter analysis. Cytogenetics involves analysis of the chromosome of cancer cells to identify genetic abnormalities, while mutational analysis detects specific genetic mutations associated with different types of blood cancers. Clinical parameters assess factors such as subject age, disease stage, and overall health. While these methods provide valuable insights, they also have significant limitations. For instance, some or all of the foregoing methods require invasive procedures like bone marrow biopsies, which are uncomfortable and carry risks for subjects. Additionally, these methods are often specific to single types of blood cancer and do not provide a comprehensive prognostic view across multiple hematologic malignancies, which may lead to fragmented and sometimes conflicting prognostic information. Furthermore, they do not adequately account for the biological diversity within and between different types of blood cancers, leading to variable prognostic accuracy.

Accordingly, the novel concepts described herein are generally directed to the development of a pan-heme prognostic classifier that uses targeted methylation sequencing of nucleic acids, such as DNA and RNA, to predict survival outcomes in subjects with hematologic malignancies. The novel classifier may leverage cell-free DNA (cfDNA) data or cfRNA data obtained from a biological sample, e.g., a liquid biopsy, such as a blood draw, to measure methylation levels, generating a beta value matrix for data analysis. In an aspect, the method may employ nested cross-validation for robust training and validation, utilizing Cox regression models and principal component analysis (PCA) for dimensionality reduction and survival prediction. Personalized risk scores may be generated and used to stratify subjects into risk categories, such as one or more of high, medium, or low risk categories.

In an aspect, the utilization of cfDNA or cfRNA obtained from blood samples or other liquid biopsies eliminates the need for invasive procedures like bone marrow aspiration, thereby significantly reducing subject discomfort and risk. Additionally, the non-invasive nature of the classifier may allow for more frequent monitoring of subjects, enabling better tracking of disease progression and response to treatment. The pan-heme classifier may provide a unified prognostic tool applicable across a wide range of hematologic malignancies, addressing the biological heterogeneity that conventional methods struggle with. By leveraging advanced methylation sequencing and data analysis techniques, the classifier may enhance the accuracy of risk stratification, leading to more precise and personalized treatment planning. Further, the classifier may uncover shared and unique biological mechanisms among different blood cancers, contributing to a more comprehensive understanding of prognostic factors across various hematological lineages.

The concepts described herein provide a variety of technical improvements, particularly in the realm of computation technology and data analysis. For instance, the techniques described herein leverage high-dimensional data and machine-learning techniques to create a comprehensive and unified risk stratification tool. The integration of these computational methods allows for the processing and analysis of large and complex datasets, leading to more precise and personalized risk assessments. Furthermore, the system leverages high-dimensional data from targeted methylation sequencing, which generates a beta value matrix representing methylation levels across numerous regions of the genome. Analyzing this data requires sophisticated computational models to identify relevant patterns and reduce dimensionality. Therefore, neither the processes needed to construct the novel classifier, nor the analytical processes that the novel classifier is configured to perform, can be performed in the human mind due to the complexity and scale of the data processing and analysis involved. Accordingly, the technical improvements provided by the disclosed concepts are not merely abstract ideas but constitute a practical application that enhances the functionality of existing prognostic tools, as evidenced by the performance improvements further illustrated and described herein (e.g., higher concordance index scores and lower p-value scores as compared to various baseline models).

The subject matter of the present disclosure will now be described more fully hereinafter with reference to the accompanying drawings, which form a part hereof, and which show, by way of illustration, specific exemplary embodiments. An embodiment or implementation described herein as “exemplary” is not to be construed as preferred or advantageous, for example, over other embodiments or implementations; rather, it is intended to reflect or indicate that the embodiment(s) is/are “example” embodiment(s). Subject matter may be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware, or any combination thereof. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” or “in some embodiments,” or “in one aspect” or “in some aspects” as used herein does not necessarily refer to the same embodiment or aspect, and the phrase “in another embodiment” or “in another aspect” as used herein does not necessarily refer to a different embodiment or aspect. It is intended, for example, that claimed subject matter include combinations of exemplary embodiments in whole or in part.

Diseases referred to herein may include cancer. For instance, non-limiting hematologic malignancies referred to herein may include b-cell lymphoma, CLL_SLL, DLBCL, essential thrombocythemia, follicular lymphoma, Hodgkin lymphoma, lymphoplasmacytic, MALT NMZL, mantle cell, MDS, MGUS, plasma cell myeloma, plasma cell neoplasm, and polycythemia vera. Additionally or alternatively, non-limiting cancer types that the concepts described herein may be applied to include, for example, breast cancer, lung cancer (e.g., non-small cell lung cancer (NSCLC)), prostate cancer, colorectal cancer, renal cancer, uterine cancer, pancreatic cancer, cancer of the esophagus, a lymphoma, head and neck cancer, ovarian cancer, a hepatobiliary cancer, a melanoma, cervical cancer, multiple myeloma, leukemia, thyroid cancer, bladder cancer, and gastric cancer. Additionally, it is also important to note that although the concepts described throughout this disclosure are made in reference to cancer, these designations are for exemplary purposes only and are not intended to be limiting. Specifically, the concepts described herein may be applicable to other disease types and other disease-detecting machine-learning classifiers.

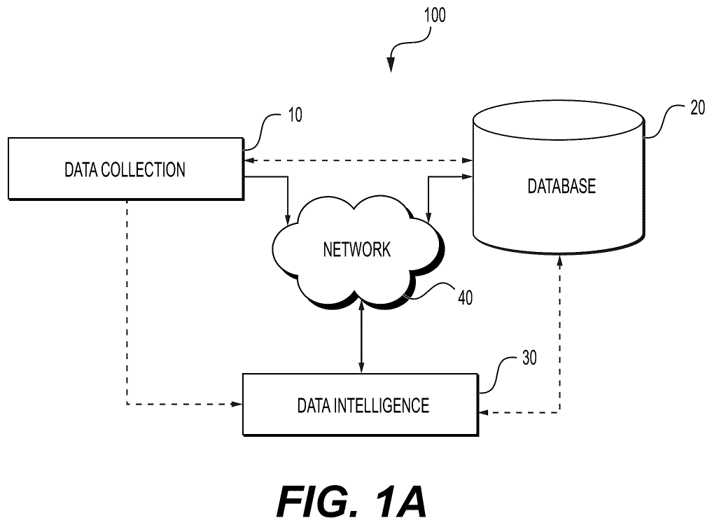

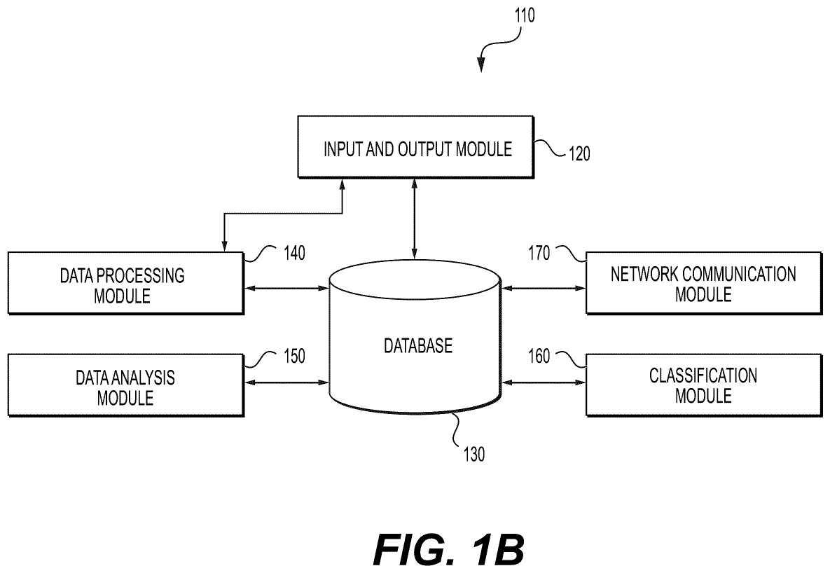

FIG. 1A depicts an exemplary system for developing and employing a prognostic classifier based on targeted methylation sequencing of, e.g., cfDNA, for survival outcome prediction in blood cancers. Exemplary system 100 includes a data collection component 10, a database 20, and device data intelligence component 30, operably connected to each other via network 40. Alternatively, or additionally, one or more of the components may be connected with another component locally without reliance on network connection; e.g., through a wired connection. In many aspects described herein, sequencing data of cell-free nucleic acids from blood samples are used to illustrate the concepts. However, one of skill in the art would understand that the current method may be applied to sequencing data of DNA, RNA, or other materials, as well from a variety of sample types, e.g., a blood sample (e.g., a serum sample, a plasma sample, a whole blood sample), a urine sample, a saliva sample, a tissue sample, a bone marrow sample, etc.

As disclosed herein, data collection component 10 may include a device or machine with which sequencing data may be generated. In some embodiments, data collection component 10 may include one or more sequencing devices or a facility that uses one or more sequencing devices to generate nucleic acid (e.g., DNA or RNA) sequence data of biological samples. In some aspects, data collection component 10 may be a database that receives sequencing information generated from one or more sequencing devices. Any suitable liquid or solid biological samples may be used for sequencing. In some embodiments, a biological sample may be cell-based, for example, one or more types of tissue. In some embodiments, a biological sample may be a sample that includes cell-free nucleic acid fragments. Examples of biological samples include, but are not limited to, a blood sample (e.g., a cfDNA sample, a cfRNA sample, a serum sample, a plasma sample, a whole blood sample), a urine sample, a saliva sample, a tissue sample, a bone marrow sample, etc. Further, although sequencing of DNA from these samples is discussed herein, RNA from these samples may alternatively or additionally be sequenced.

Examples of sequencing data may include, but are not limited to, sequence read data of targeted genomic locations, partial or whole genome sequencing data of the genome represented by nucleic acid fragments in cell-free or cell-based samples, partial or whole genome sequencing data including one or more types of epigenetic modifications (e.g., methylation), or combinations thereof.

Data acquired by the data collection component 10 may be transferred to database 20 via network 40 or a local or network connection. In some embodiments, data collection component 10 may alternatively receive data from one or more sequencing devices. In some embodiments, the collected data may be analyzed by data intelligence component 30, via network 40 or a local or network connection. FIG. 1B depicts exemplary functional modules that may be implemented to perform tasks of data intelligence component 30.

FIG. 1B depicts an exemplary computer system 110 for providing a prognostic classifier for survival outcome prediction in blood cancers. Exemplary system 110 achieves such functionalities by implementing, on one or more computer devices, user input and output (I/O) module 120, memory or database 130, data processing module 140, data analysis module 150, classification module 160, network communication module 170, and any other functional modules that may be needed for carrying out a particular task (e.g., an error correction or compensation module, a data compression module, etc.). As disclosed herein, user I/O module 120 may further include an input sub-module, such as a keyboard, and an output sub-module, such as a display (e.g., a printer, a monitor, or a touchpad). In some embodiments, all functionalities may be performed by one computer system. In some embodiments, the functionalities are performed by more than one computer system.

Also disclosed herein, a particular task may be performed by implementing one or more functional modules. In particular, each of the enumerated modules itself may, in turn, include multiple sub-modules. For example, data processing module 140 may include a sub-module for data quality evaluation (e.g., for discarding very short sequence reads or sequence reads including obvious errors), a sub-module for normalizing numbers of sequence reads that align to different regions of a reference genome, a sub-module to compensate/correct guanine-cytosine (GC) biases, a sub-module for matching data associated with a cancer sample with other data associated with one or more non-cancer samples, etc.

In some embodiments, a user may use I/O module 120 to manipulate data that is available either on a local device or can be obtained via a network connection from a remote service device or another user device. For example, I/O module 120 may allow a user, e.g., via a keyboard, a mouse, or a touchpad, to initiate or perform data analysis via a graphical user interface (GUI). In some embodiments, a user may manipulate data via voice control. In some embodiments, user authentication may be required before a user is granted access to the data being requested. In some embodiments, user I/O module 120 may be used to manage various functional modules. For example, a user may request via user I/O module 120 input data while an existing data processing session is in process. A user may do so by selecting a menu option or type in a command discretely without interrupting the existing process. In another example, a user may utilize user I/O module 120 to set various thresholds, configure sample matching settings, and/or provide other instructions to computer system 110 that dictate how data may be analyzed. As disclosed herein, a user may use any type of input to direct and control data processing and analysis via I/O module 120.

In some embodiments, system 110 further comprises a memory or database 130. In some embodiments, database 130 comprises a local database that may be accessed via user I/O module 120. In some embodiments, database 130 comprises a remote database that may be accessed by user I/O module 120 via network connection. In some embodiments, database 130 is a local database that stores data retrieved from another device (e.g., a user device or a server). In some embodiments, memory or database 130 may store data retrieved in real-time from internet searches. In some embodiments, database 130 may send data to and receive data from one or more of the other functional modules, including, but not limited to, a data collection module (not shown), data processing module 140, data analysis module 150, classification module 160, network communication module 170, and etc.

In some embodiments, database 130 may be a database local to the other functional modules. In some embodiments, database 130 may be a remote database that may be accessed by the other functional modules via wired or wireless network connection (e.g., via network communication module 170). In some embodiments, database 130 may include a local portion and a remote portion.

In some embodiments, system 110 comprises a data processing module 140. Data processing module 140 may receive data from I/O module 120 or database 130. In some embodiments, data processing module 140 may perform standard data processing algorithms, such as one or more of noise reduction, signal enhancement, normalization of counts of sequence reads, correction of GC bias, etc. In some embodiments, data processing module 140 may be configured to detect and measure methylation signatures, and specifically abnormal and/or differentially methylated features.

In some embodiments, system 110 comprises a data analysis module 150. In some embodiments, data analysis module 150 includes identifying and treating systematic errors in sequencing data, as described in connection with data processing module 140.

In some embodiments, system 110 comprises a classification module 160, which may embody a “machine-learning model” or “trained classifier.” As used herein, a “machine-learning model” or “trained classifier” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration. In some aspects, the machine-learning model may be trained on a combination of real and synthetic sample data.

The execution of the machine-learning model may include deployment of one or more machine-learning techniques, such as k-nearest neighbors, linear regression, logistic regression, random forest, gradient boosted machine (GBM), deep learning, a deep neural network, and/or any other suitable machine-learning technique that solves problems in the field of Natural Language Processing (NLP). Supervised, semi-supervised, and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

In an exemplary use case, a machine-learning model may be trained to analyze data from a test sample from a test subject whose status with respect to a medical condition is unknown and subsequently classifies the unknown test sample from the test subject based on the likelihood of the subject fitting into a particular category (e.g., positive or negative for a disease condition; one or more of high, medium, or low risk for developing a disease condition or a prognosis for a disease condition, and/or prognostic factors associated with the disease, such as a survival probability risk stratification, etc.). In some embodiments, the one or more parameters may include a binomial probability score that is calculated based on logistic regression analysis. As disclosed herein, the binomial probability score may correspond to the likelihood of a subject having a certain medical condition, such as cancer, and/or prognostic factors, such as a survival probability risk stratification, e.g., into a high or low risk classification. For example, a score of over a predefined threshold may indicate that the subject associated with a test sample is more likely to have cancer than not have cancer, or more likely than not to fall into a high risk category. In some embodiments, the one or more parameters may include a sequencing or methylation data distribution pattern correlating with the presence of cancer or falling into a given risk category, which may be indicative of survival timelines. A subject associated with a test sample having sequencing or methylation data with a pattern resembling the cancer pattern to a sufficient degree may be predicted as having cancer, or a subject associated with a test sample having sequencing or methylation data with a pattern resembling a high, low, or medium risk prognostic classification. In some embodiments, a sequencing or methylation data distribution pattern may be identified in connection with a specific type of cancer, determining a tissue of origin or cancer signal origin, thus allowing a test sample to be classified as indicative of a certain cancer type. In some embodiments, a sequencing or methylation data distribution pattern may be identified in connection with a specific prognostic, risk, or survival categorization, thus allowing a subject associated with a test sample to be categorized as falling into a certain prognosis, risk, or survival category.

As disclosed herein, network communication module 170 may be used to facilitate communications between a user device, one or more databases, and any other suitable system or device through a wired or wireless network connection. Any communication protocol/device may be used, including, without limitation, a modem, an Ethernet connection, a network card (wireless or wired), an infrared communication device, a wireless communication device, and/or a chipset (such as a Bluetooth™ device, an 802.11 device, a WiFi device, a WiMax device, cellular communication facilities, etc.), a near-field communication (NFC), a Zigbee communication, a radio frequency (RF) or radio-frequency identification (RFID) communication, a PLC protocol, a 3G/4G/5G/LTE based communication, and/or the like. For example, a user device having a user interface platform for processing/analyzing CHIP-related methylation signature data may communicate with another user device with the same platform, a regular user device without the same platform (e.g., a regular smartphone), a remote server, a physical device of a remote IoT local network, a wearable device, a user device communicably connected to a remote server, and etc.

The functional modules described herein are provided by way of example. It will be understood that different functional modules may be combined to create different utilities. It will also be understood that additional functional modules or sub-modules may be created to implement a certain utility.

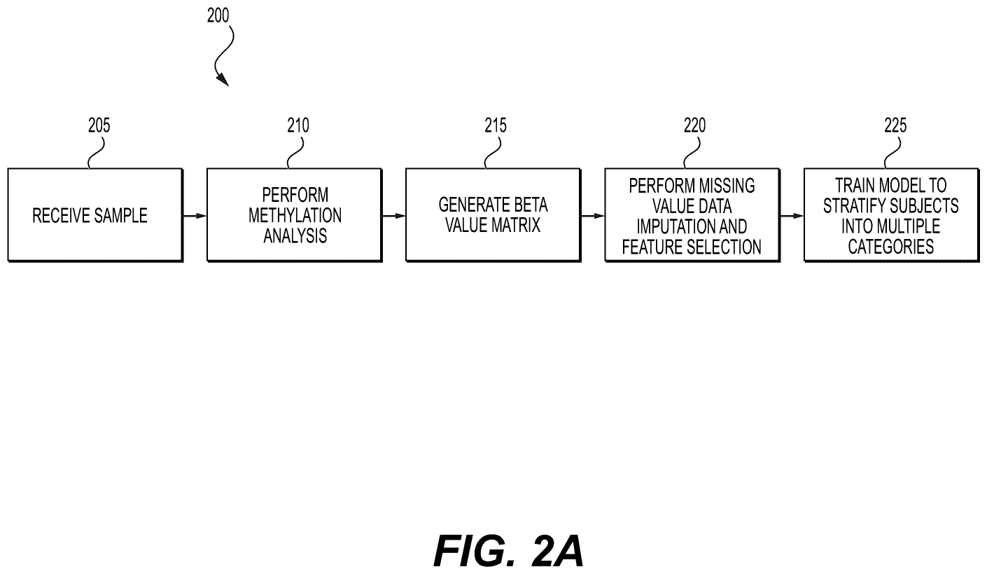

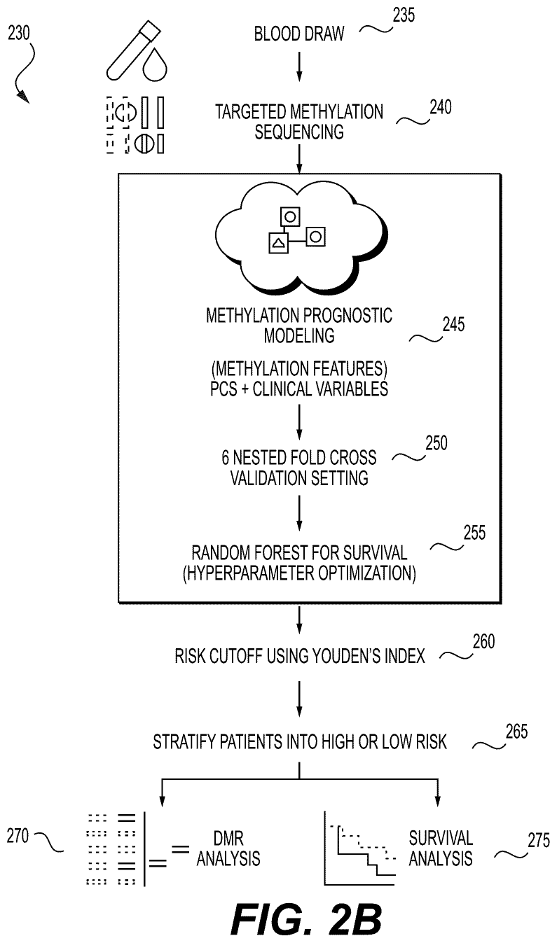

Referring now to FIG. 2A, an exemplary workflow 200 is described for providing a prognostic classifier designed to predict survival outcomes for subjects with various hematologic malignancies (e.g., leukemia, lymphoma, and myeloma, or others described herein). Aspects of the exemplary workflow 200 may be performed in accordance with some or all components described in FIGS. 1A and 1B.

At step 205, data may be received from subjects diagnosed with various hematologic malignancies. Biological samples, such as blood samples, may be received from subjects diagnosed with one of a range of blood cancers, including, e.g., b-cell lymphoma, CLL_SLL, DLBCL, essential thrombocythemia, follicular lymphoma, Hodgkin lymphoma, lymphoplasmacytic, MALT NMZL, mantle cell, MDS, MGUS, plasma cell myeloma, plasma cell neoplasm, polycythemia vera, and others not explicitly listed here. These malignancies may be chosen to represent a broad spectrum of hematologic disorders, enabling the classifier to address the diverse biological characteristics inherent to different blood cancers. In an aspect, the received biological samples may have been obtained by drawing peripheral blood from subjects, from which cfDNA may be subsequently extracted. cfDNA may be a preferred source for non-invasive testing as it circulates in the bloodstream and reflects the genetic and epigenetic alterations present in the tumor cells. In an aspect, this extraction may be performed used standardized protocols to ensure the integrity and quality of the cfDNA to promote accurate methylation analysis.

In an aspect, once the cfDNA has been extracted from the received biological samples, a determination may be made about which samples are evaluable. This selection process may involve one or more selection criteria to ensure that the data used for training and validating the prognostic classifier is of high quality and reliability. For instance, in one aspect, evaluable samples may be those that have sufficient cfDNA quantity and quality, with low levels of degradation and contamination. This ensures that the methylation data that is subsequently obtained is accurate and reliable. Additionally or alternatively, in another aspect, evaluable samples may be required to have adequate sequencing coverage to ensure comprehensive detection of methylation patterns across the targeted genomic regions. Samples with insufficient coverage may miss critical methylation events, leading to incomplete or biased data. Additionally or alternatively, cfDNA samples may be required to be accompanied by complete and accurate clinical data, including one or more of (subject demographics (e.g., age, sex), clinical diagnosis, treatment history, and/or survival outcomes. Incomplete clinical data may compromise the integrity of the prognostic model and its validation. Additionally or alternatively, in cases in which multiple samples are received from the same subject, technical replicates may be evaluated for consistency, with those samples showing significant variability or discrepancies being excluded to main data quality.

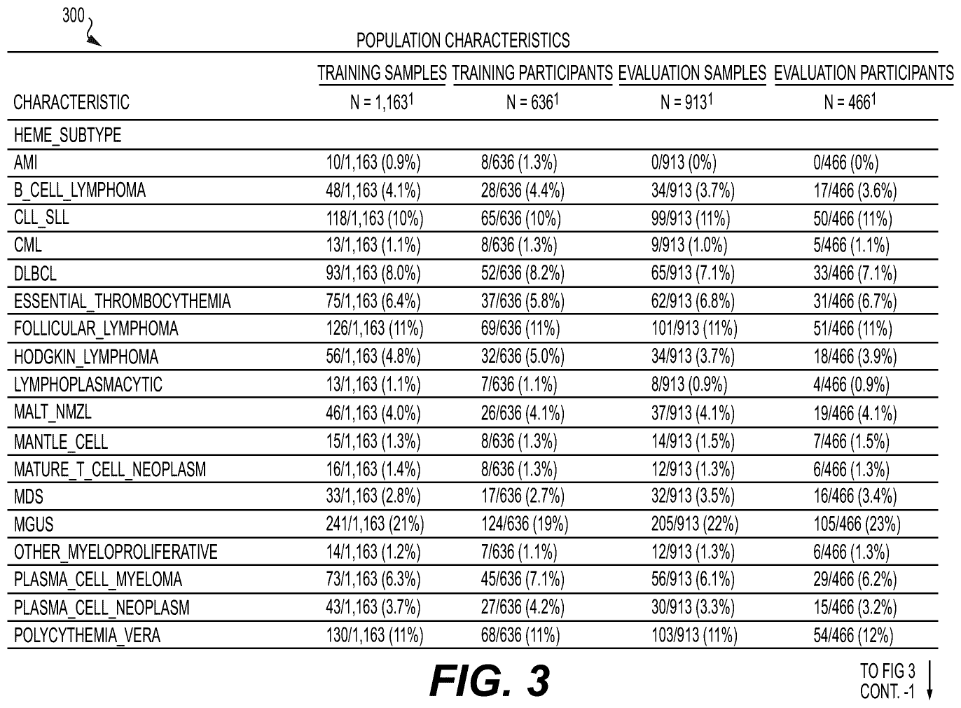

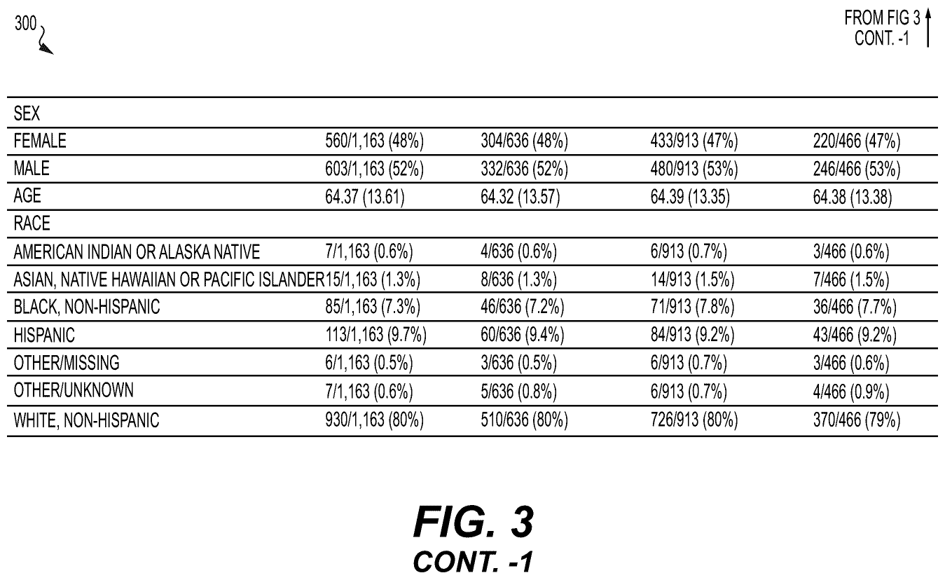

FIG. 3 depicts diagram 300 that presents a detailed breakdown of the population characteristics used in the study to develop the prognostic classifier. Diagram 300 shows the distribution of different hematologic malignances among the training and evaluation samples. The study utilized a total of 1163 training samples, which included subjects with multiple samples collected over time, resulting in 636 unique training participants. Not all of the collected samples could be used for evaluating the performance of the classifier due to the criteria described above. Accordingly, from the total population, 913 samples were deemed evaluable (i.e., indicating that they met the necessary quality and completeness criteria for inclusion in the analysis). These 913 samples corresponded to 466 unique evaluation participants.

Referring back to FIG. 2A, at step 210, targeted methylation sequencing may be performed on the extracted cfDNA from the biological samples. This process involves the measurement of DNA methylation levels across selected regions of the genome, which are known to be relevant for hematologic malignancies. In an aspect, the cfDNA extracted from the samples may undergo bisulfate conversion, which is a chemical treatment that converts unmethylated cytosines to uracils while leaving methylated cytosines unchanged. This conversion allows for the differentiation between methylated and unmethylated cytosines during sequencing. The bisulfite-treated cfDNA may then be subjected to targeted sequencing, focusing on predefined genomic regions of interest. These regions may be chosen based on their known or potential association with hematologic malignancies, ensuring that the data collected is relevant for prognostic analysis. Targeted sequencing enables high-depth coverage of these specific regions, allowing for accurate quantification of methylation levels. In an aspect, advanced sequencing platforms, such as next-generation sequencing (NGS), may be employed to perform the targeted methylation sequencing. These platforms generate large amounts of data by reading the sequences of bisulfite-treated cfDNA fragments. The sequencing reads are then aligned to a reference genome, and the methylation status of each cytosine in the targeted regions is determined by comparing the sequence data to the bisulfite-converted reference.

At step 215, a beta value matrix may be generated from the targeted methylation sequencing data. In an aspect, the output of the process described above may be a beta value matrix, in which each value represents the methylation level of a specific cytosine in a given genomic condition. Beta values range from 0 to 1, with 0 indicating complete unmethylation and 1 indicating complete methylation. This matrix may serve as the foundation for subsequent data analysis, including dimensionality reduction, model training, and risk score generation.

At step 220, in an aspect, raw methylation data may contain missing values and high dimensionality, which may adversely affect the performance and accuracy of the prognostic model if not properly addressed. Accordingly, to handle missing data, two approaches may be employed: a non-imputation approach and an imputation approach. In the non-imputation approach, missing beta values may simply be ignored. This method assumes that the available data is sufficient for effective analysis, although it risks excluding potentially valuable information. Conversely, the imputation approach involves filling in missing values with either mean or median imputation values calculated from the training data. Before imputation, regions with more than 20% missing values are removed to maintain data integrity. This approach ensures that the dataset remains as complete as possible, leveraging all available information to improve the robustness of the model.

In an aspect, the beta value matrix typically contains a vast number of features, which can lead to overfitting and computation inefficiencies if not properly managed. Accordingly, regardless of the approach utilized to address the missing data, a dimensionality reduction process, such as PCA, may be employed to address the issue. PCA may be used to reduce the dimensionality of the data by transforming the original high-dimensional feature space into a smaller set of uncorrelated principal components. These components capture the most significant variance in the data, effectively summarizing the essential information while discarding noise and redundancies. By retaining the top principal components, the model may focus on the most informative features, thereby enhancing its predictive accuracy and computational efficiency. In some aspects, the preprocessing stage may also involve normalizing the data to ensure that features are on a comparable scale. Normalization may involve scaling the beta values to a standard range or distribution, so that no single feature disproportionally influences the model's predictions.

At step 225, the values in the generated beta value matrix may be utilized to train and validate a machine learning model. In an aspect, although a variety of different types of machine learning models may be utilized to develop the prognostic classifier, the specific type of model described herein is a random survival forest (RSF) model. The RSF model belongs to a category of machine learning models known as ensemble learning methods. Ensemble learning combines multiple individual models to produce a more robust and accurate prediction than any single model alone. The most well-known ensemble method is the Random Forest, which is primarily used for classification and regression tasks. RSF is an extension of the Random Forest model tailored for survival analysis. Survival analysis models may be utilized when the outcome of interest is the time until an event occurs (e.g., death), and they must handle censored data (e.g., instances where the event has not occurred for some subjects during the study period).

As an initial step, the entire dataset may be split into training and validation sets. To achieve this, in an aspect, a six-fold nested cross-validation approach may be employed. Nested cross-validation is a technique that helps mitigate overfitting and provides an unbiased estimate of model performance. In this approach, the dataset may be divided into six folds, of which five folds are used for training the model, and the sixth fold is used for validation. This process may be repeated six times, with each fold taking its turn as the validation set. The goal is to use every data point for both training and validation, providing a comprehensive evaluation of the model's performance.

To prevent information leakage, samples from the same subject may be assigned to the same fold. This precaution may be necessary because using samples from the same subject in both training and validation sets may lead to inflated performance estimates, as the model may inadvertently learn subject-specific characteristics rather than generalizable patterns. Accordingly, by keeping all samples from the same subject within the same fold, the model's ability to generalize to new, unseen subjects is preserved. Additionally, in an aspect, each fold may be balanced based on several key characteristics so that the training and validation sets are representative of the entire dataset. The balancing criteria may include sex (e.g., promoting an equal representation of male and female subjects), heme subtype (e.g., maintaining a proportional distribution of different hematologic malignancies across the folds), evaluable status (e.g., including only samples that meet the quality and completeness criteria for evaluable status), age category (e.g., distributing age groups evenly to prevent age-related biases), and overall survival days (e.g., categorizing survival outcomes and ensuring they are evenly represented in each fold).

Within each training iteration, multiple decision trees may be constructed. For each tree, a bootstrap sample (e.g., a random sample with replacement) may be drawn from the training data (e.g., the five folds combined). This bootstrap sampling introduces variability so that each tree is slightly different. In an aspect, at each node of a tree, a random subset of features may be selected to determine the best split. The best split at each node is chosen based on a criterion that maximizes the separation of survival times, often using the log-rank test statistic. This process may continue until a stopping criterion is met (e.g., a minimum node size or maximum tree depth is realized). In an aspect, data points not included in the bootstrap sample for a tree (e.g., “out-of-bag (OOB) samples”) may be used internally to validate each tree and provide an unbiased estimate of the model's performance. The survival predictions for the validation folds are aggregated, and the concordance index (“c-index”) is calculated to evaluate the model's ability to rank survival times correctly. In an aspect, key hyperparameters, such as the number of variables randomly sampled at each split (“mtry”) and the minimum node size, are tuned using an inner cross-validation loop. Different combinations of hyperparameters are tested to find the best-performing set that maximizes the c-index. In an aspect, a grid search may be employed to systematically explore the hyperparameter space and identify the optimal values. In an aspect, once all decision trees are constructed, their individual survival predictions are aggregated. Each tree provides a survival function, and these functions are averaged to produce the final survival prediction for each subject.

After completing the cross-validation process and identifying the best hyperparameters, the final RSF model may be refitted on the entire dataset using these optimal parameters. As a result, the model benefits from all available data and is fine-tuned for the optimum performance. In an aspect, the final RSF model may be configured to stratify subjects into multiple categories, e.g., high-risk vs. low-risk groups or high, medium, and low-risk groups, based on their risk scores. The thresholds for these groups may be determined using the Youden's index, which balances sensitivity and specificity. In an aspect, the performance of the final RSF model may be evaluated using the c-index and other relevant metrics, so that the model provides accurate and reliable survival predictions.

In an aspect, survival outcomes in the context of hematologic malignances may refer to the length of time a subject lives after the time from the initial blood draw. In an aspect, these outcomes may typically be measured in terms of overall survival, which is the length of time from diagnosis, or the start of treatment, until death from any cause. In an aspect, these ranges may include short-term survival (e.g., under 1 year), medium-term survival (1-5 years), and long-term survival (e.g., more than 5 years).

As mentioned above, subjects may be categorized into high-risk, medium-risk, and low-risk groups. In an aspect, subjects categorized as high risk may have the highest probability of adverse outcomes, such as disease progression or mortality within a specified time frame. Clinically, these subjects may exhibit rapid disease progression, resistance to standard treatments, and poor overall survival rates (e.g., less than 1 year). These subjects may need frequent monitoring to manage complications and detect disease progression early. The high-risk classification may aid clinicians in prioritizing these subjects for more aggressive or experimental therapies and closer monitoring. In an aspect, subjects classified as medium risk may have an intermediate probability of adverse outcomes. This group may exhibit some genetic and epigenetic changes associated with the disease but to a lesser extent than the high-risk group. Subjects classified into the medium-risk group may have overall survival rates that are generally better than those in the high-risk group but worse than those in the low-risk group (e.g., 1 to 5 years). These subjects may need regular monitoring to adjust treatments based on their response. In an aspect, subjects classified as low risk may have the lowest probability of adverse outcomes. This group may typically exhibit fewer or less severe genetic and epigenetic abnormalities. Clinically, these subjects may experience slower disease progression, respond well to standard treatments, and have higher overall survival rates. These subjects may require less frequent monitoring compared to high-risk subjects but still need regular check-ups to control disease progression. Accordingly, the low-risk classification may allow clinicians to consider less aggressive treatment options and less frequent monitoring, focusing on maintaining quality of life. Although three categories (medium, high, and low risk) are described herein, other more or fewer categories may be used to classify subjects based on their prognosis, and different time spans or other qualifications may be used. For example, if two life-expectancy categories are used, the categories may only include high and low risk, which may be divided into, e.g., those with less than a 5-year survival expectancy (high risk), and those with greater than a 5-year survival expectancy (low risk).

Referring now to FIG. 2B, flow diagram 230 is provided that complements the disclosure associated with exemplary flow diagram 200. Aspects of the exemplary workflow 230 may be performed in accordance with some or all components described in FIGS. 1A and 1B.

In an aspect, at step 235, blood samples may be received. The blood samples may have been collected from subjects diagnosed with hematologic malignances. cfDNA may be obtained from the drawn blood, which may contain genetic and epigenetic information from tumor cells. At step 240, the cfDNA extracted from the blood samples may undergo targeted methylation sequencing. This process may involve bisulfite conversion, which differentiates between methylated and unmethylated cytosines, followed by sequencing to measure DNA methylation levels across specific genomic regions.

At steps 245, 250, and 255, feature selection, cross-validation, model training and hyperparameter tuning may occur in the construction of a methylation prognostic model. More particularly, with respect to step 245, methylation features, along with PCs derived from a beta value matrix, may be combined with clinical variables to form the input data for modeling. At step 250, a six-fold nested cross-validation approach is employed to mitigate overfitting and provide an unbiased estimate of model performance. In this approach, the dataset may be divided into six folds, where five folds are used for training and one fold for validation. This may allow each data point to be used for training and validation. At step 255, a random forest model, tailored for survival analysis, may be used to train the prognostic classifier. This RSF model is configured to handle censored data and predict survival times. Hyperparameter optimization may be conducted to identify the best-performing model parameters, which enhances the model's predictive accuracy.

At step 260, Youden's index may be utilized to determine the optimal cutoff points for stratifying subjects into different risk categories. More particularly, in this experiment, cutoff values were derived by applying Youden's index to 5-year survival receiver operator characteristic (ROC). Based on the identified risk cutoff points, subjects may, at step 265, be stratified into high-risk and low-risk groups, although more cutoff points and groups may be used. This stratification may help in understanding the prognosis and tailoring treatment plans accordingly.

At optional step 270, the high-risk and low-risk groups may be further analyzed to identify significant DMRs. This analysis may help in understanding the molecular differences between the risk groups and the potential impact on gene regulation and cancer progression. In an aspect, differential methylation analysis of cfDNA samples from high-risk and low-risk groups was performed using beta-binomial regression with an arcsine link function to model the region-level count data, adjusting for sex. In an aspect, hypothesis testing was performed using a Wald's test for each region. In an aspect, the DMRs were considered significant if the q-value was under 0.05 and the absolute value of the beta-binomial model delta coefficients was greater than 0.1. In an aspect, KEGG pathway enrichment analysis may be performed using the closest genes to each significant hyper- or hypo-DMR using a hypergeometric test, as further discussed herein.

At step 275, survival analysis may be conducted to evaluate the model's performance in predicting survival outcomes. The analysis may provide insights into the survival probabilities of subjects in different risk categories, e.g., as compared to one or more other models (e.g., various types of baseline models). In an aspect, the log-rank test was utilized to examine survival differences, and Kaplan-Meier plots were utilized to visually represent the survival distribution across different risk groups, as further discussed and illustrated herein.

TABLE 1

| Models | Variables Included | Overall C-Index |
|---|---|---|
| Baseline | Clinical Variables | 0.706 |
| | | [0.641, 0.768] |
| Baseline_TF | Clinical Variables + | 0.721 |
| | log2(TF) | [0.660, 0.781] |
| Baseline_pcancer | Clinical Variables + | 0.739 |
| | P_cancer | [0.678, 0.798] |
| Clinical_methylation_feature | Clinical Variables + | 0.7408 |
| | Methylation PCs | [0.6759, 0.7960] |

Table 1 above provides a comparison of various models trained to predict survival outcomes in subjects with hematologic malignancies. Each model includes different sets of variables and uses different methods for handling methylation data. In an aspect, the baseline model may include the clinical variables, such as one or more of heme subtype, race, age, sex, highest clinical stage, body mass index (BMI), smoking status, and drinking status. There is no beta value imputation for this model (because it does not use methylation data) and the overall c-index was determined to be 0.706, indicating the model's ability to discriminate between different survival times. In an aspect, the baseline_TF model includes the same clinical variables present in the baseline model plus the logarithm of tumor fraction (log₂ (TF)). There is no beta value imputation for this model, and the overall c-index was determined to be 0.721, showing an improvement over the baseline model by incorporating tumor fraction data. In an aspect, the baseline_pcancer model includes the same clinical variables present in the baseline model plus the p-cancer score. There is no beta value imputation for this model, and the overall c-index was determined to be 0.721, further improving the prediction accuracy compared to the baseline and baseline_TF models. In an aspect, the clinical methylation feature model, the construction of which is described above, includes the same clinical variables present in the baseline model plus a set a set of principal components (PCs), e.g., the top 10 PCs, derived from the methylation data. In some aspects, no beta value imputation may be performed for this model, and the overall c-index score was determined to be 0.749, representing the best-performing model among the four listed. In other aspects, a median imputation approach may be utilized to address the missing beta values. Based on the collective data in Table 1, it can be seen that incorporating methylation features may significantly enhance a model's predictive accuracy compared to models relying solely on clinical variables or other methylation-derived features.

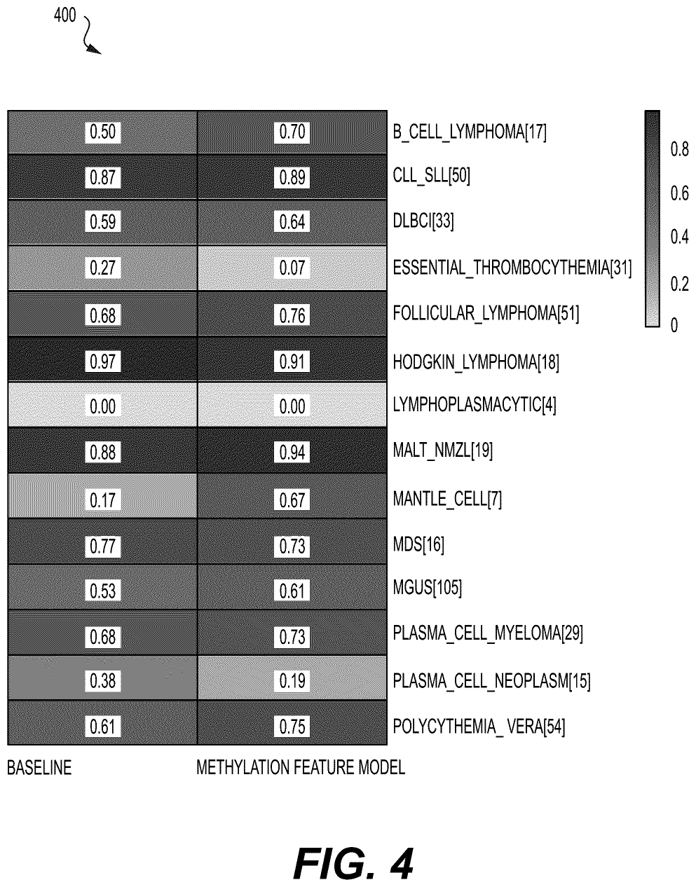

Referring now to FIG. 4, diagram 400 presents a complimentary visualization of the data presented in Table 1. More particularly, diagram 400 presents a breakdown of the performance of the baseline model across various hematologic malignancy subtypes. In an aspect, each cell in the heatmap represents the performance of a model for a specific subtype, measured by the c-index metric, with the values color-coded from 0 (yellow) to 1 (dark purple). The subtypes include a range of hematologic malignancies, and the numbers in brackets next to each subtype indicate the number of unique subjects with that specific indication. The clinical methylation feature model, which includes methylation features with no imputation, shows notable improvement in several indications compared to the baseline model This is evident in subtypes such as b-cell lymphoma, CLL/SLL, MGUS, and polycythemia vera. Diagram 400 indicates that incorporating methylation data may enhance the predictive accuracy of the prognostic model for these subtypes. This suggests that methylation features provide valuable additional information that compliments traditional clinical variables.

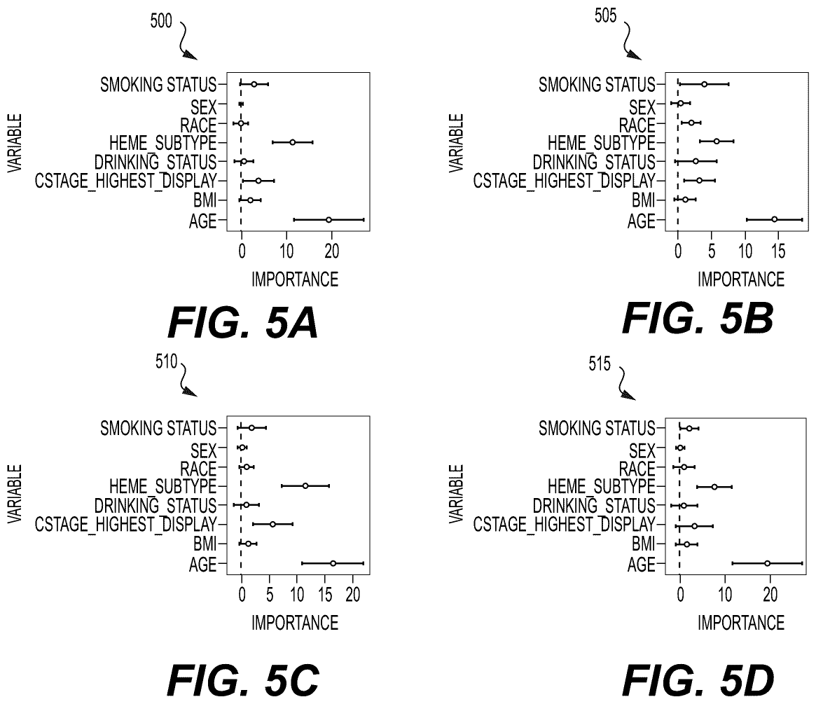

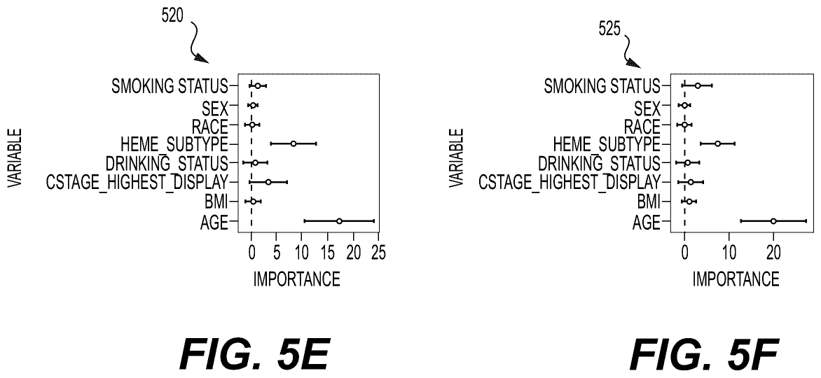

Referring now to FIGS. 5A-5F, variable importance plots 500-525 are provided for the baseline RSF model across six nested cross-validation folds. Each plot corresponds to one of the six training folds, highlighting the importance of different clinical variables such as smoking status, sex, race, heme subtype, drinking status, highest clinical stage (e.g., cancer stage I, II, III, or IV), BMI, and age. These plots help in understanding which variables are most predictive in the model. More particularly, these plots indicate how much each variable contributes to the predictive power of the model. A higher importance score means the variable has a greater impact on the model's predictions. In an aspect, each of the boxes in the plots represent the interquartile range (IQR), with the line inside the box indicating the median importance score. The whiskers extend to the minimum and maximum values within the 95% confidence interval. Examination of plots 500-525 reveal that age and heme subtype are among the most predictive variables across all six folds.

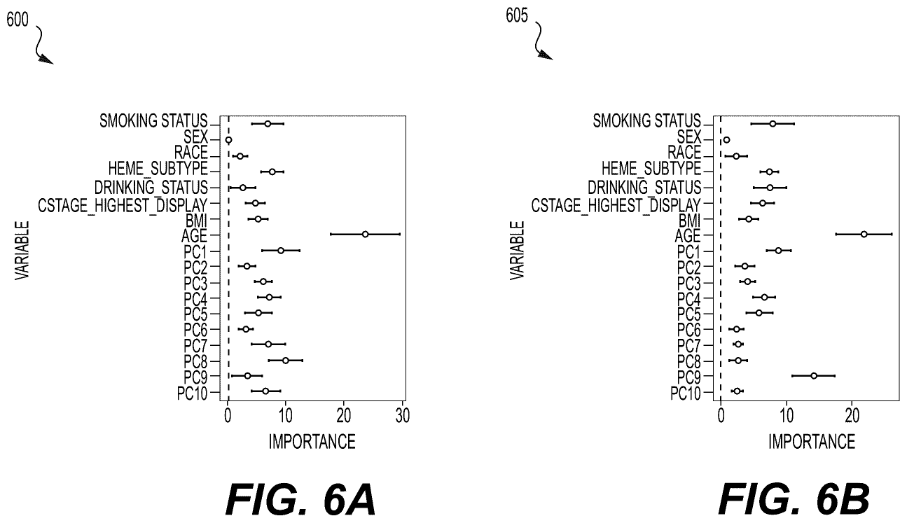

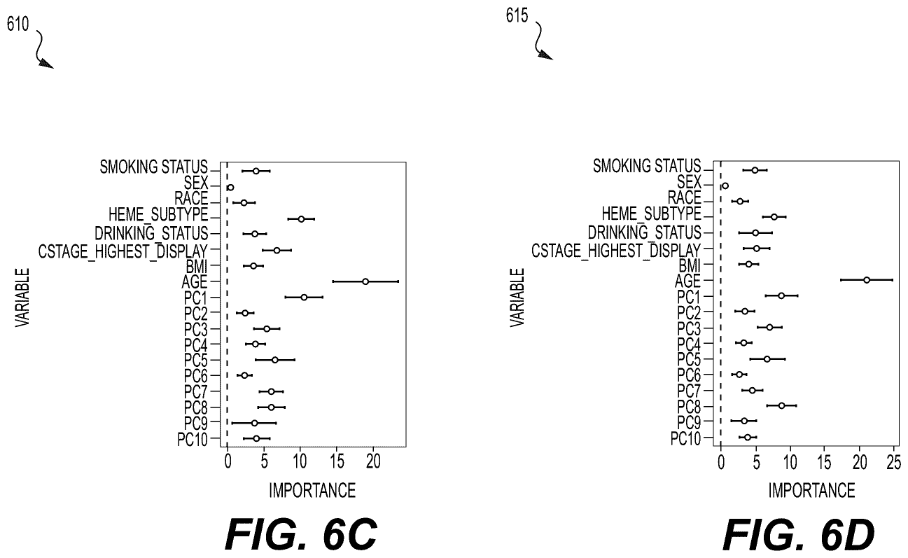

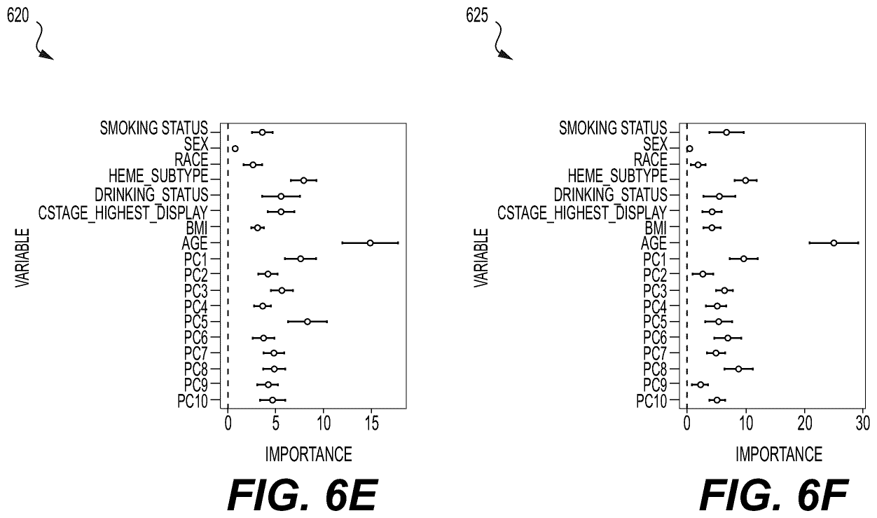

Referring now to FIGS. 6A-6F, variable importance plots 600-625 are provided for the clinical methylation feature model, which integrates clinical variables with the top 10 PCs derived from methylation data. Similar to FIGS. 5A-5F, each plot corresponds to one of the sex nested cross-validation folds. Examination of the plots 600-625 reveals that across all six folds, age remains the most predictive variable, demonstrating high importance scores. Additionally, the PCs derived from the methylation data show significant predictive power. In several folds, some PCs have comparable or even higher importance scores than heme subtype, indicating that methylation features contribute meaningfully to the model's predictions.

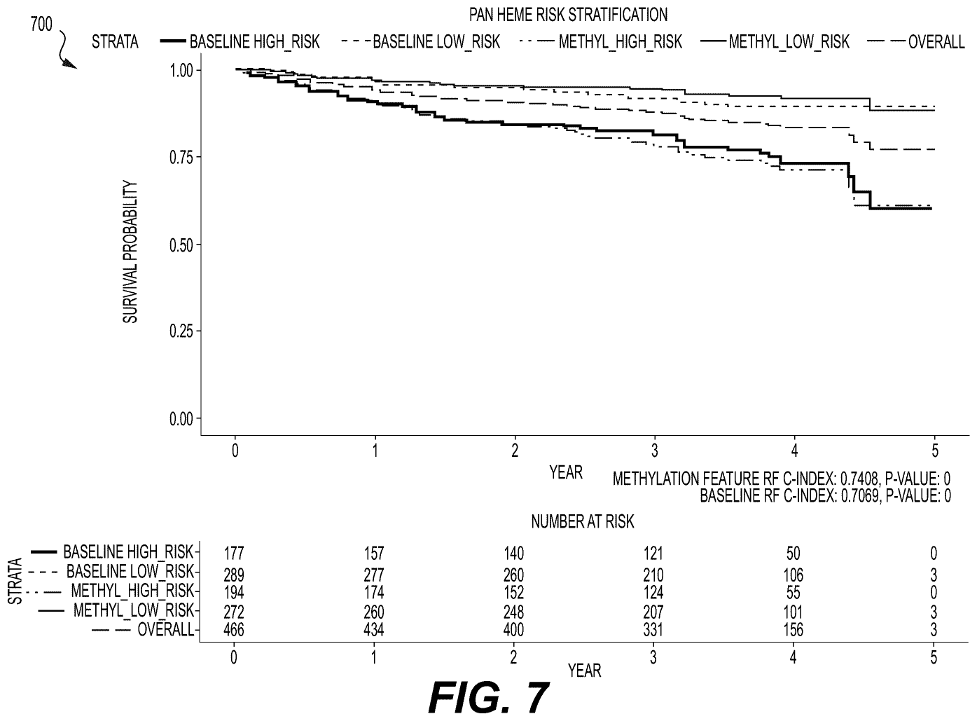

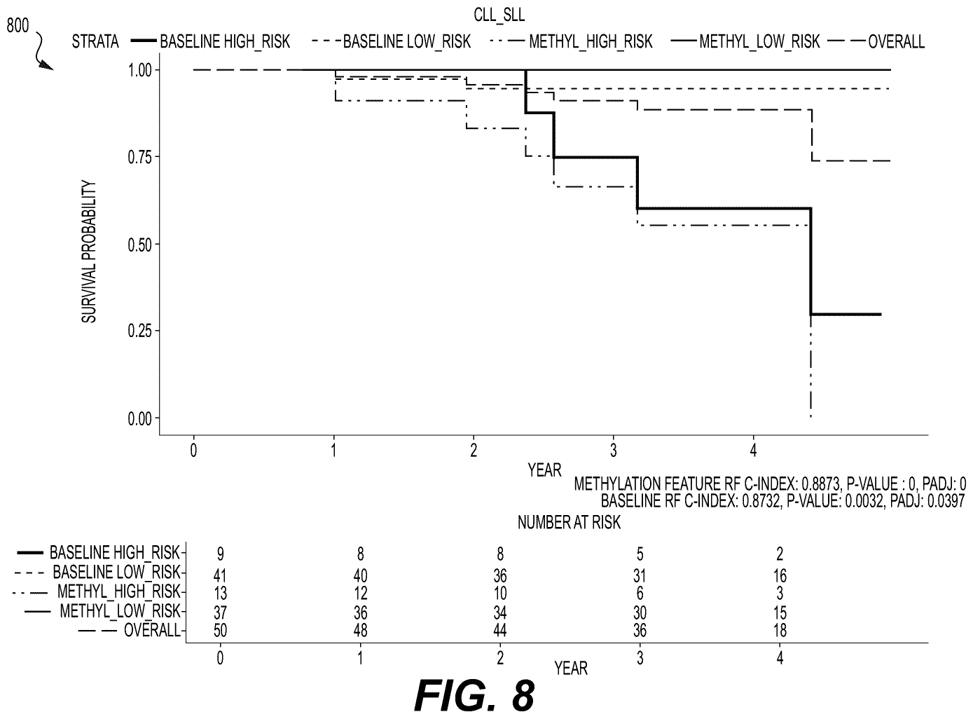

FIGS. 7-18 present a series of Kaplan-Meier plots for a multitude of cancer types, with each plot comparing the survival curves generated by the clinical methylation feature model with those from the baseline model. Using the Youden index, subjects are categorized into high-risk and low-risk groups. The legend associated with each plot identifies the baseline high risk group, the baseline low risk group, the clinical methylation feature high risk group, the clinical methylation feature low risk group, and the overall survival for all subjects. Collectively, these plots provide a visual representation of survival probabilities over time for different risk groups, helping to assess the effectiveness of the proposed model in stratifying subjects by risk.

FIG. 7 is directed to assessing pan-heme risk classification, meaning across a number of different types of blood cancer (e.g., those cancer types indicated in FIG. 3). With respect to plot 700 in FIG. 7, the clinical methylation feature model achieves an overall c-index of 0.7408, indicating good predictive accuracy for survival outcomes. Examination of plot 700 reveals that the stratification is statistically significant, with a p-value of 0, indicating a strong distinction between the high and low-risk groups. This suggests that the proposed model effectively separates subjects into meaningful risk categories. Although the baseline model has a comparable p-value, the survival curves for the clinical methylation feature model are more distinctly separated than those of the baseline model, indicating better stratification. Additionally, the baseline model has a lower c-index (0.7069) compared to the clinical methylation feature model (0.7408), demonstrating that the latter has better predictive accuracy.

FIG. 8 presents a Kaplan-Meier plot for chronic lymphocytic leukemia small lymphocytic lymphoma (CLL SLL). In plot 800 in FIG. 8, the clinical methylation feature model achieves an overall c-index of 0.8873 indicating strong predictive accuracy for survival outcomes in CLL SLL subjects. Additionally, plot 800 reveals that the stratification is highly significant, with a p-value of 0, indicating a strong distinction between the high and low-risk groups. Plot 800 reveals that the survival curves for the clinical methylation feature model show greater separation between high and low-risk groups compared to the baseline model. The baseline model's p-value of 0.0032, while still significant, is less robust. Additionally, the baseline model has a c-index of 0.8732, compared to the 0.8873 of the clinical methylation feature model, demonstrating that the latter has better predictive accuracy.

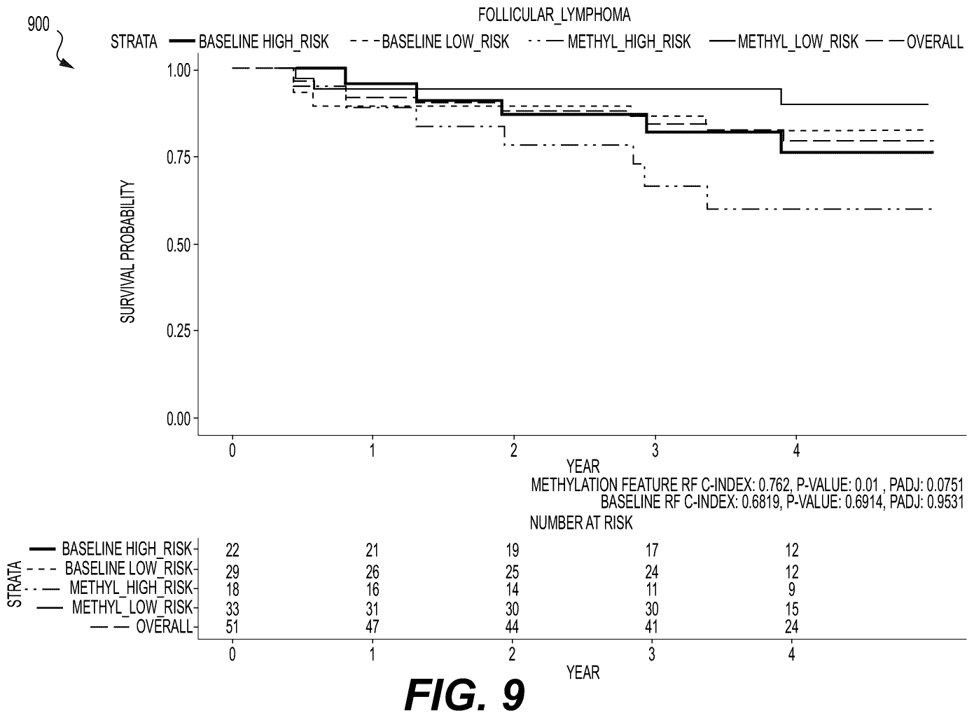

FIG. 9 presents a Kaplan-Meier plot for follicular lymphoma. In plot 900 in FIG. 9, the clinical methylation feature model achieves an overall c-index of 0.762, indicating good accuracy for survival outcomes in follicular lymphoma subjects. Additionally, plot 900 reveals that the stratification is statistically significant, with a p-value of 0.01, indicating a meaningful distinction between the high and low-risk groups, suggesting that the clinical methylation feature model can effectively separate subjects into different risk categories. Plot 900 also reveals that the survival curves for the clinical methylation feature model show greater separation between high and low-risk groups compared to the baseline model. The baseline model's p-value is 0.6914, which is not significant. Additionally, the baseline model has a c-index of 0.6819, compared to the 0.762 of the clinical methylation feature model, demonstrating that the latter has better predictive accuracy.

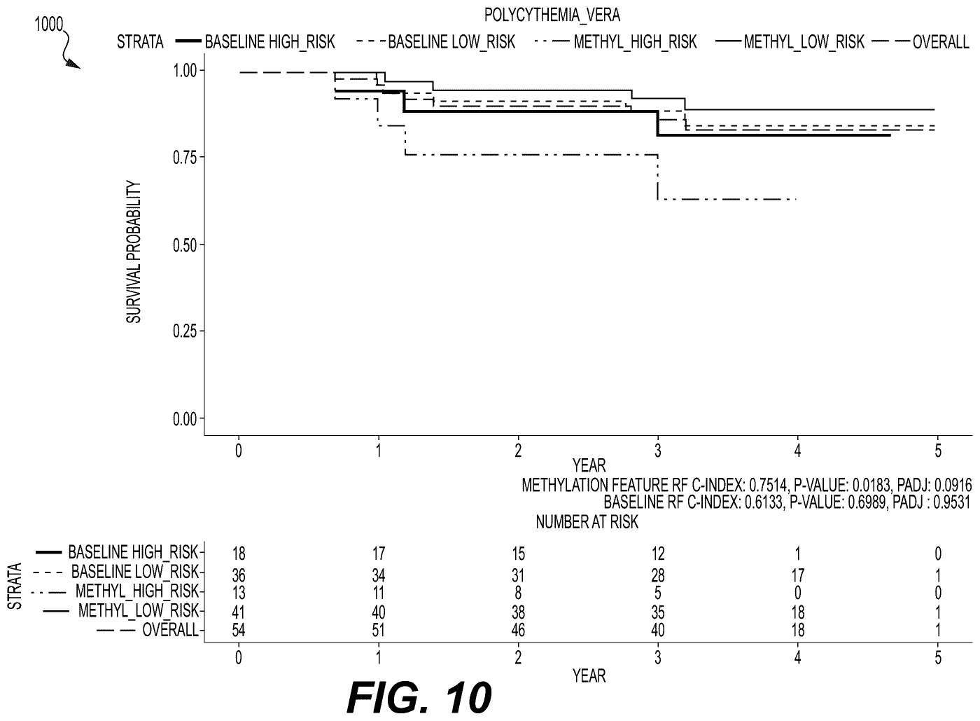

FIG. 10 presents a Kaplan-Meier plot for polycythemia vera. In plot 1000 in FIG. 10, the clinical methylation feature model achieves an overall c-index of 0.7514, indicating good accuracy for survival outcomes in polycythemia vera subjects. Additionally, plot 1000 reveals that the stratification is statistically significant, with a p-value of 0.0183, indicating a meaningful distinction between the high and low-risk groups, suggesting that the clinical methylation feature model can effectively separate subjects into different risk categories. Plot 1000 also reveals that the survival curves for the clinical methylation feature model show greater separation between high and low-risk groups compared to the baseline model. The baseline model's p-value is 0.6989, which is not significant. Additionally, the baseline model has a c-index of 0.6133, compared to the 0.7514 of the clinical methylation feature model, demonstrating that the latter has better predictive accuracy.

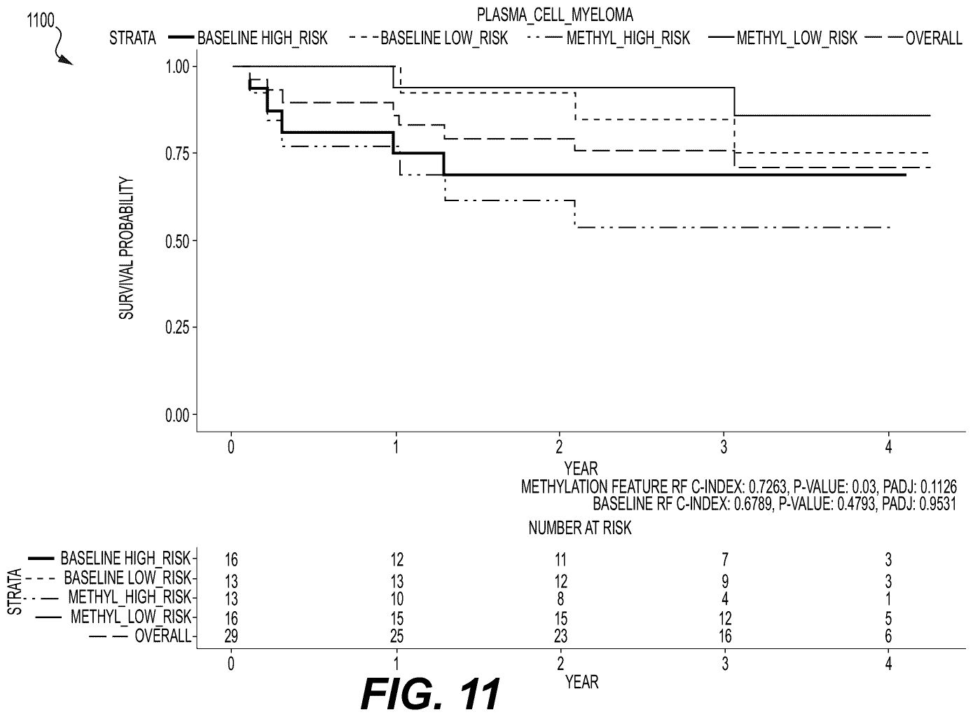

FIG. 11 presents a Kaplan-Meier plot for plasma cell myeloma. In plot 1100 in FIG. 11, the clinical methylation feature model achieves an overall c-index of 0.7263, indicating good accuracy for survival outcomes in plasma cell myeloma subjects. Additionally, plot 1100 reveals that the stratification is statistically significant, with a p-value of 0.03 indicating a meaningful distinction between the high and low-risk groups, suggesting that the clinical methylation feature model can effectively separate subjects into different risk categories. Plot 1100 also reveals that the survival curves for the clinical methylation feature model show greater separation between high and low-risk groups compared to the baseline model. The baseline model's p-value is 0.4793, which is not significant. Additionally, the baseline model has a c-index of 0.6789, compared to the 0.7263 of the clinical methylation feature model, demonstrating that the latter has slightly better predictive accuracy.

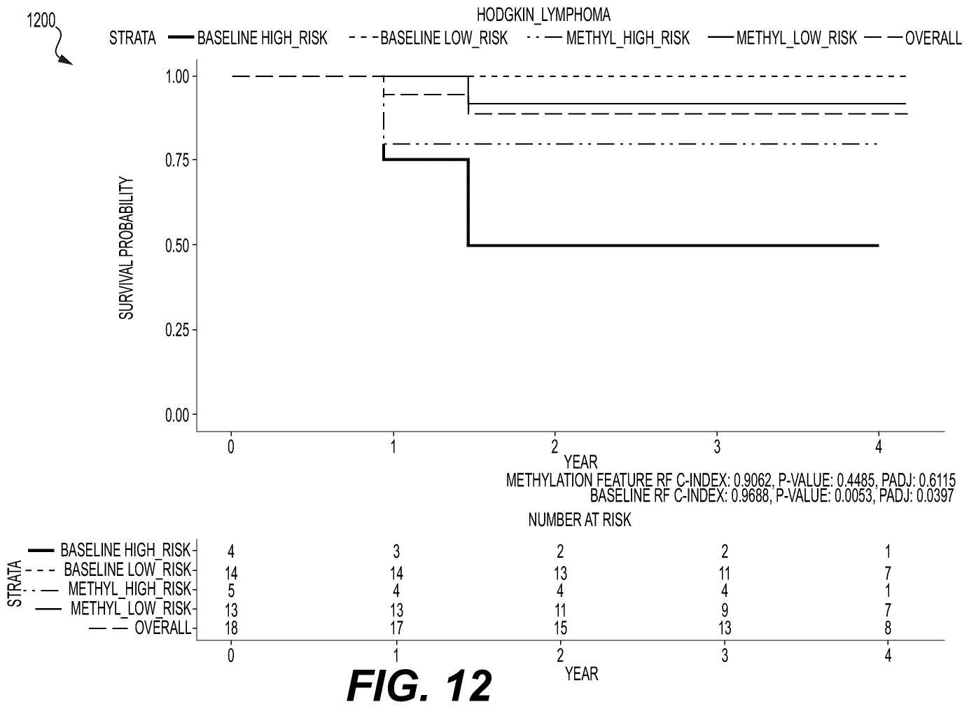

FIG. 12 presents a Kaplan-Meier plot for Hodgkin lymphoma. In plot 1200 in FIG. 12, the clinical methylation feature model achieves an overall c-index of 0.9062, indicating very high predictive accuracy for survival outcomes in Hodgkin lymphoma subjects. Additionally, plot 1200 reveals that the stratification is not significant, with a p-value of 0.4485. The limited number of samples (as shown by the small numbers at risk) may contribute to the higher p-value. Plot 1200 also reveals that the baseline model exhibits better stratification between high and low-risk groups than the clinical methylation feature model. The baseline model's p-value is 0.0053, indicating significant stratification. Additionally, the baseline model has a higher c-index (0.9688) compared to the 0.9062 c-index of the clinical methylation feature model, demonstrating that the former has slightly better predictive accuracy.

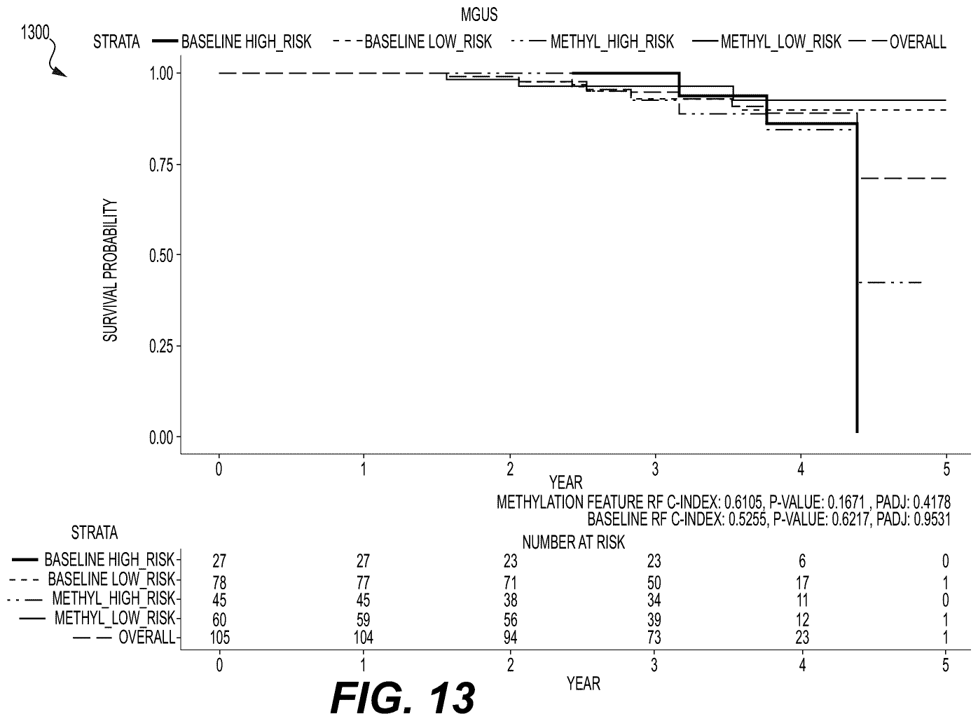

FIG. 13 presents a Kaplan-Meier plot for monoclonal gammopathy of undetermined significance (MGUS). In plot 1300 in FIG. 13, the clinical methylation feature model achieves an overall c-index of 0.6105, indicating moderate predictive accuracy for survival outcomes in MGUS subjects. Additionally, plot 1300 reveals that the stratification is not significant, with a p-value of 0.1671. This may be explained because MGUS is a pre-cancer condition and its progression to cancer can take a longer time. The current dataset spans only 5 years, which may not be sufficient to capture the model's full prognostic potential, thus contributing to the less significant results. Plot 1300 also reveals that the survival curves for the clinical methylation feature model and the baseline model both show some separation between high and low-risk groups. The baseline model's p-value is 0.6217, indicating no significant stratification. Additionally, the baseline model has a lower c-index of 0.5255 compared to the 0.6105 c-index of the clinical methylation feature model, demonstrating that the latter has better predictive accuracy.

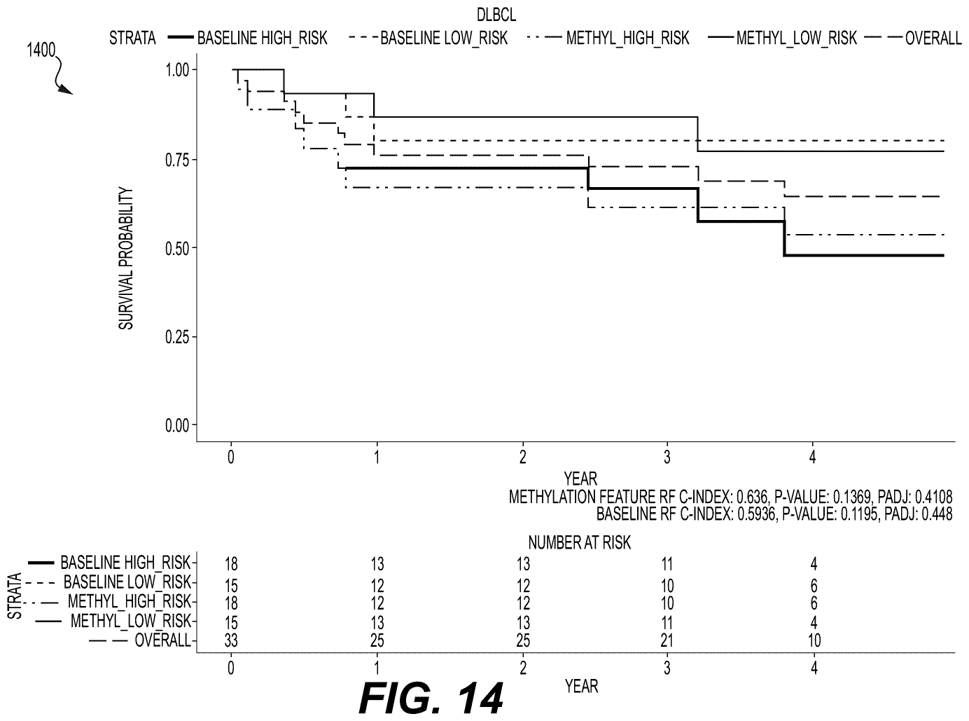

FIG. 14 presents a Kaplan-Meier plot for diffuse large B-cell lymphoma (DLBCL). In plot 1400 in FIG. 14, the clinical methylation feature model achieves an overall c-index of 0.636, indicating moderate accuracy for predicting survival outcomes in DLBCL subjects. Additionally, plot 1400 reveals that the stratification shows a trend toward separation, but is not statistically significant with a p-value of 0.1369. This lack of significance may be attributed to the small sample size, which limits the power to detect differences between groups. Plot 1400 also reveals that the baseline model exhibits lower stratification between high and low-risk groups, with the clinical methylation feature model exhibiting slightly better predictive power compared to the baseline model. The baseline model's p-value is 0.1195, which is not significant. Additionally, the baseline model has a c-index of 0.5936, which is slightly lower than the c-index of 0.636 of the clinical methylation feature model. This suggests that the inclusion of methylation features slightly enhances the predictive accuracy over the baseline model for DLBCL based on initial studies.

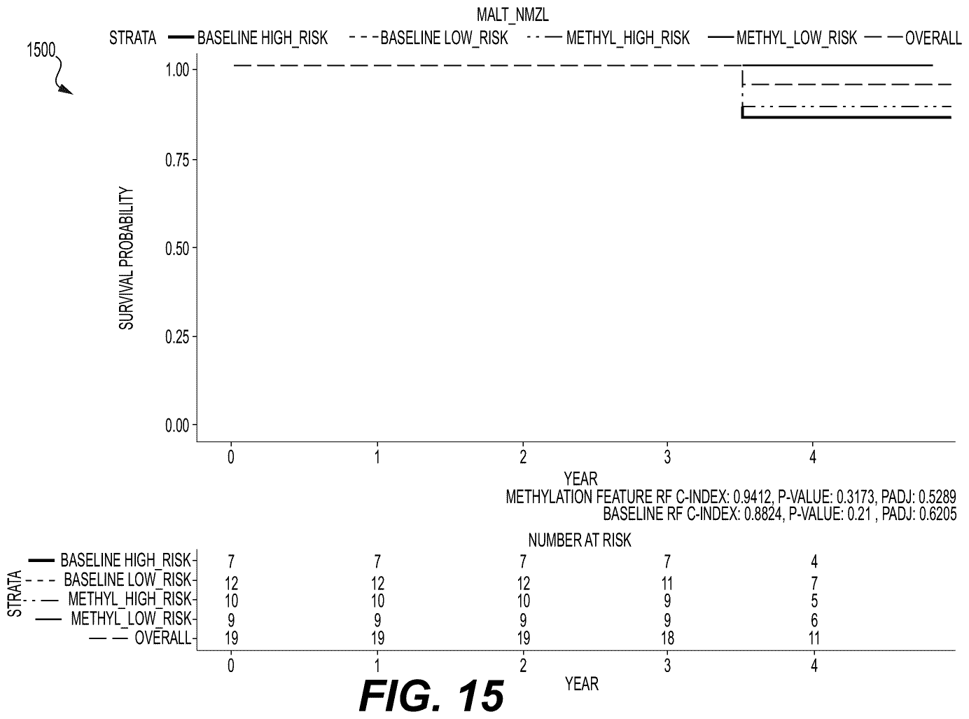

FIG. 15 presents a Kaplan-Meier plot for Mucosa-Associated Lymphoid Tissue (MALT) Nodal Marginal Zone Lymphoma (NMZL). In plot 1500 in FIG. 15, the clinical methylation feature model achieves an overall c-index of 0.9412, indicating very high accuracy for predicting survival outcomes in MALT NMZL subjects. Additionally, plot 1500 reveals that the stratification is not statistically significant, with a p-value of 0.3173. The limited sample size may have an effect on the statistical power, making it challenging to achieve significant results. Plot 1500 also reveals that the clinical methylation feature model exhibits better stratification between high and low-risk groups than the baseline model. More particularly, the baseline model's p-value is 0.21, which is not significant. Additionally, the baseline model has a c-index of 0.8824, which is lower than the c-index of 0.9412 of the clinical methylation feature model, demonstrating that the latter has slightly better predictive accuracy.

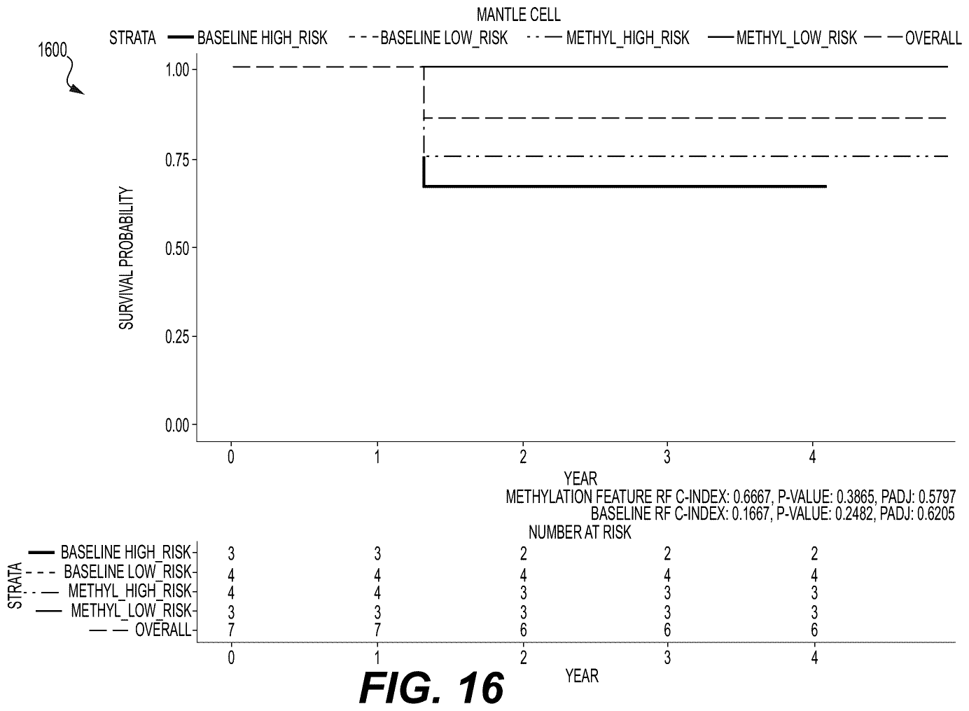

FIG. 16 presents a Kaplan-Meier plot for mantle cell lymphoma. In plot 1600 in FIG. 16, the clinical methylation feature model achieves an overall c-index of 0.6667, indicating moderate accuracy for predicting survival outcomes in mantle cell lymphoma subjects. Additionally, plot 1600 reveals that the stratification is not statistically significant, with a p-value of 0.3865. The very limited sample size likely affected the statistical power, making it difficult to achieve significant results. Plot 1600 also reveals that both models show similar stratification between high and low-risk groups, with no substantial improvement in performance from the clinical methylation feature model compared to the baseline model. The baseline model's p-value is 0.2482, which is also not significant. Additionally, the baseline model has a very low c-index of 0.167, which is much lower than the 0.6667 c-index of the clinical methylation feature model. This significant difference suggests that the inclusion of methylation features in the clinical methylation feature model enhances the predictive accuracy over the baseline model, which may be borne out by testing on larger sample sizes.

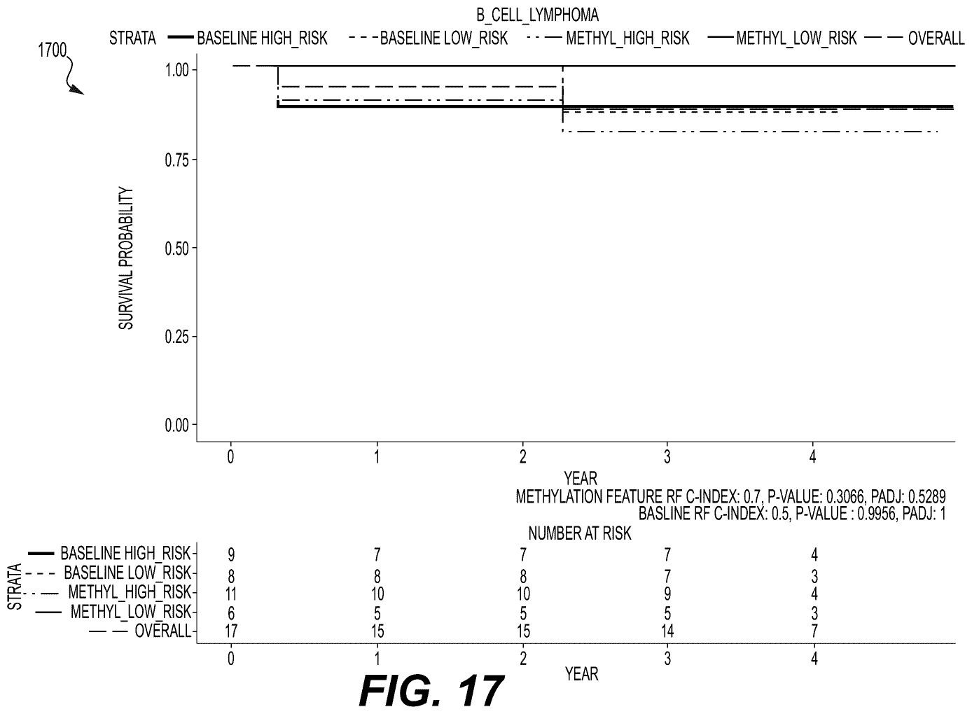

FIG. 17 presents a Kaplan-Meier plot for B-cell lymphoma. In plot 1700 in FIG. 17, the clinical methylation feature model achieves an overall c-index of 0.7, indicating moderately high accuracy for predicting survival outcomes in B-cell Lymphoma subjects. Additionally, plot 1700 reveals that the stratification is not statistically significant, with a p-value of 0.3066. Again, the limited sample size likely affected the statistical power, making it difficult to achieve significant results. Plot 1700 also reveals that both models show some separation between high and low-risk groups, but the clinical methylation feature model performed better. The baseline model's p-value is 0.9956, which is also not significant. Additionally, the baseline model has a c-index of 0.50, which is much lower than the 0.70 c-index of the clinical methylation feature model. This significant difference suggests that the inclusion of methylation features in the clinical methylation feature model enhances the predictive accuracy over the baseline model, which may be borne out by testing on larger sample sizes.

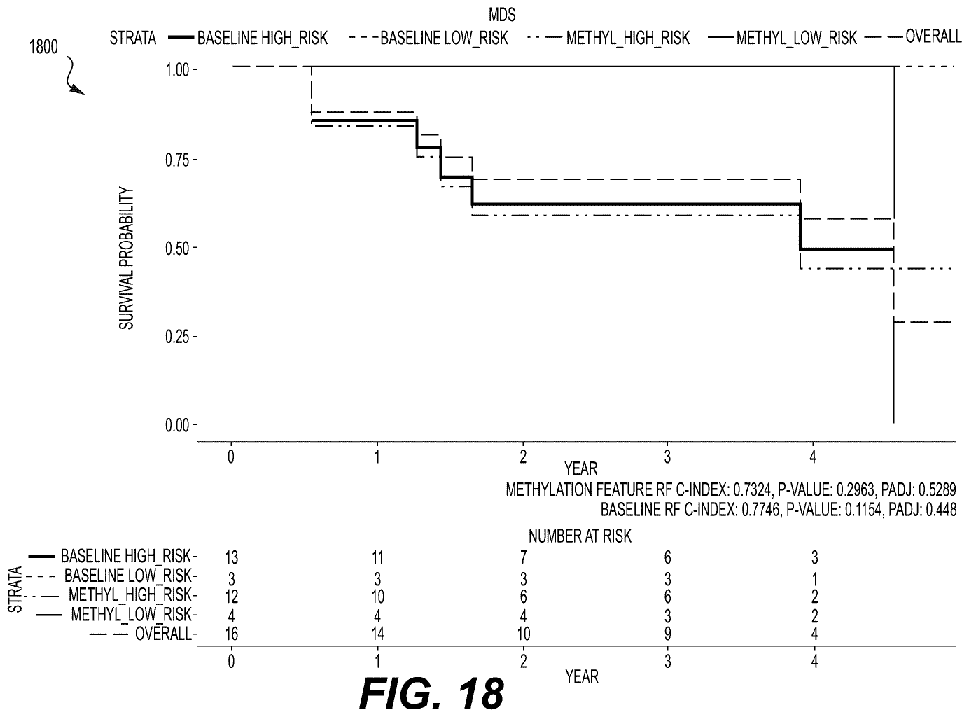

FIG. 18 presents a Kaplan-Meier plot for Myelodysplastic Syndrome (MDS). In plot 1800 in FIG. 18, the clinical methylation feature model achieves an overall c-index of 0.7324, indicating high accuracy for predicting survival outcomes in MDS subjects. Additionally, plot 1800 reveals good performance up to year 4, but the p-value of 0.2963 is not significant. The non-significance is primarily due to the survival probabilities in the last year (between 4-5 years), where the number of at-risk subjects decreases significantly. Plot 1800 also reveals that both models show some separation between high and low-risk groups, but the clinical methylation feature model performed slightly better. The baseline model's p-value is 0.1154, which is also not significant. Additionally, the baseline model has a c-index of 0.7746, which is a slight difference compared to the 0.7324 c-index of the clinical methylation feature model. This slight difference suggests that the inclusion of methylation features in the clinical methylation feature model does not provide an improvement in the predictive accuracy over the baseline model.

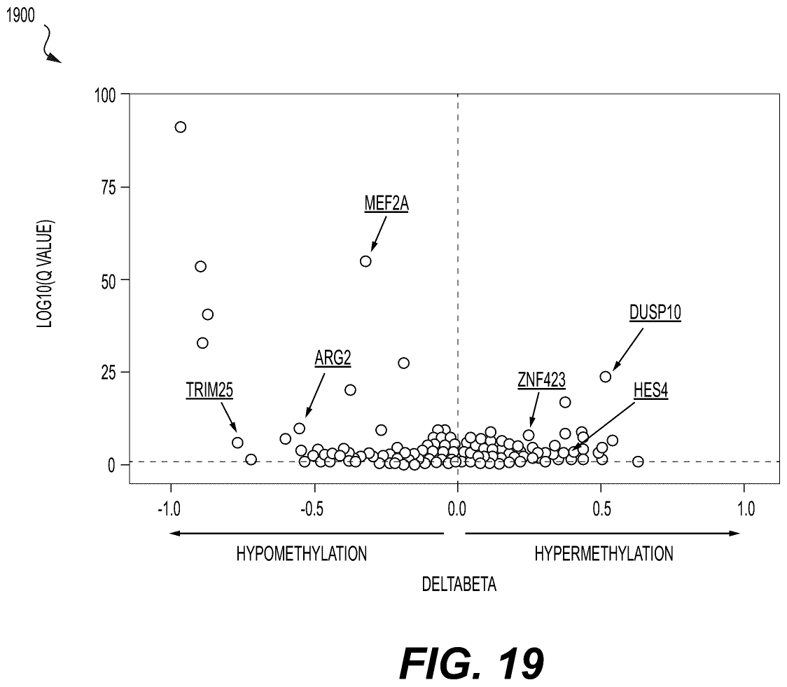

FIG. 19 presents a volcano plot 1900 that illustrates significant differentially methylated regions (DMRs) identified between high-risk and low-risk participant groups in a pan-heme context. The x-axis represents the difference in methylation levels (beta values) between high-risk and low-risk groups. Positive values indicate hypermethylation in the high-risk group, while negative values indicate hypomethylation. The y-axis represents the statistical significance of the differential methylation. Higher values indicate more significant differences. Plot 1900 provides a visual representation of the differences in DNA methylation levels between these two groups. Each point on plot 1900 represents a significant DMR, suggesting that there are distinct methylation patterns between high-risk and low-risk groups, which may be used as biomarkers for risk stratification. In an aspect, identifying these significant DMRs may help in developing prognostic classifiers to distinguish between high-risk and low-risk subjects. More particularly, significant DMRs may provide insights into the underlying epigenetic mechanisms driving the malignancy. Understanding which genes are differentially methylated may help to elucidate pathways involved in tumor progression, metastasis, and response to treatment. In plot 1900, a subset of these DMRs are highlighted. For instance, hypermethylated genes may include ZNF423, DUSP10, and HES4, whereas hypomethylated genes may include MEF21, ARG2, and TRIM25.

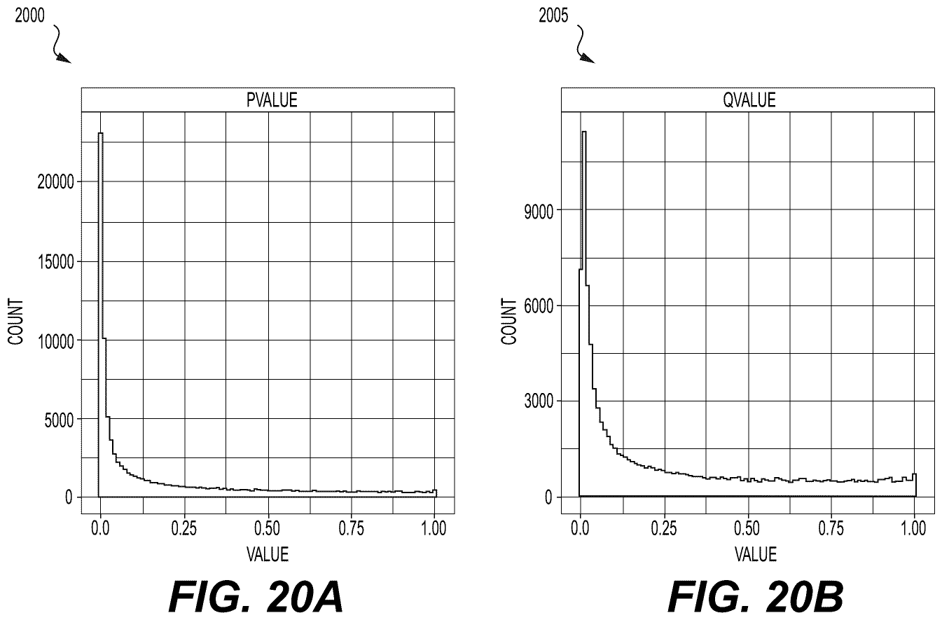

FIGS. 20A and 20B illustrate histograms 2000 and 2005, respectively, which collectively present the results of DMR analysis between high risk (N=113) and low-risk (N=109) participant groups. DMRs were considered significant if the q-value was less than 0.05, and the absolute value of beta-binomial model delta coefficients was greater than 0.2. The number of significant DMRs is 10946 (10.61%). Histogram 2000 in FIG. 20A plots the p-values from the statistical tests for differential methylation on the x-axis against the count of DMRs with the corresponding p-value on the y-axis. Histogram 2000 shows a high number of DMRs with very low p-values, indicating a significant number of regions with statistically significant differential methylation between high-risk and low-risk groups. Histogram 2005 in FIG. 20B plots the q-values (which are adjusted p-values that account for multiple testing) on the x-axis against the count of DMRs with the corresponding q-value on the y-axis. Similar to histogram 2000, histogram 2005 shows a high number of DMRs with very low q-values, confirming the significance of the methylation differences after adjusting for multiple comparisons.

FIG. 21 presents bar chart 2100, which illustrates the total length of significant DMRs in various genic features. The x-axis represents different genic features where significant DMRs are located. These features include promotor region, FpUTR region, coding sequence (CDS), 3pUTR region, other non-coding exons, intron, and none (e.g., regions that do not fall into the above categories). The y-axis represents the total length of the significant DMRs in base pairs (bp) for each genic feature. Examination of chart 2100 indicates that the largest total length of significant DMRs is found in intron regions, totaling 2,379,127 bp. This indicates that introns have substantial methylation changes that may be relevant for gene regulation.

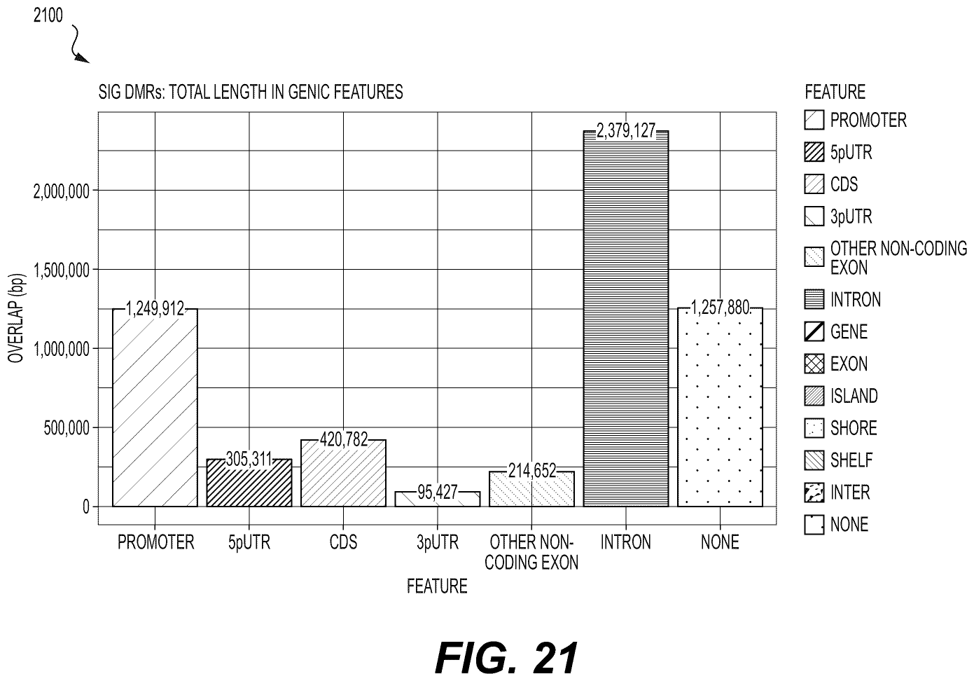

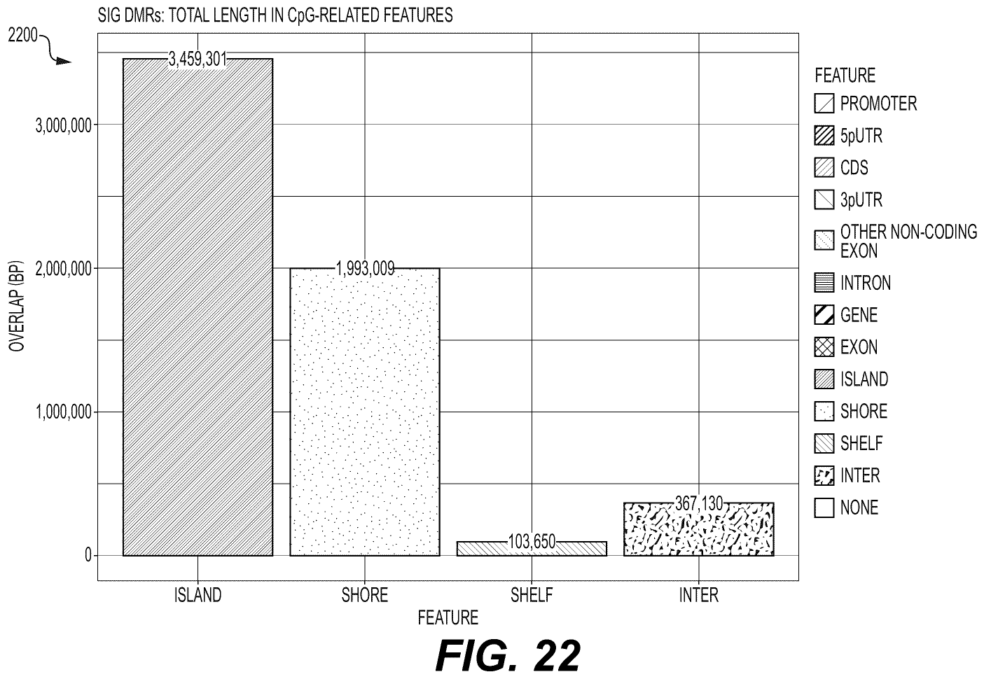

FIG. 22 presents bar chart 2200, which illustrates the total length of significant DMRs in various CpG-related features. The x-axis represents the different CpG-related features where significant DMRs are located. These features include island regions, shore regions, shelf regions, and inter regions. The y-axis represents the total length of the significant DMRs in bp for each CpG-related feature. Examination of chart 2200 indicates that the largest total length of significant DMRs is found in CpG islands. Here the length refers to the combined length of all input regions overlapping with CpG island. As CpG islands are often located in the promotor regions of genes, methylation changes in these regions may influence gene expression. More particularly, hypermethylation in CpG islands may lead to gene silencing, while hypomethylation may lead to gene activation.

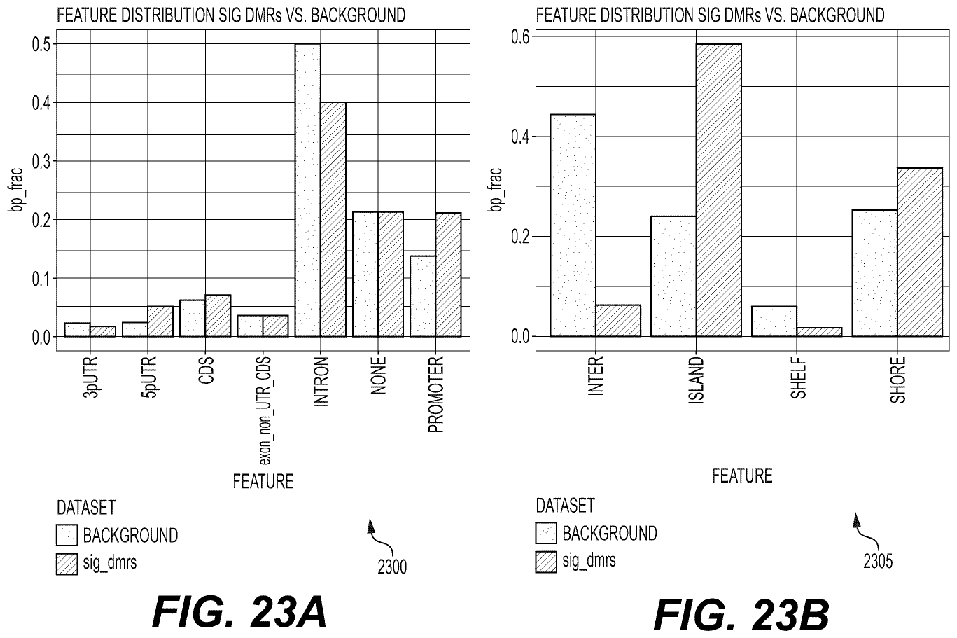

FIGS. 23A and 23B present bar charts 2300 and 2305 that compare the distribution of significant DMRs to the background in terms of genic and CpG-related features, respectively. Examination of chart 2300 reveals that features 3pUTR, 5pUTR, CDS, and exon non-coding regions show only minor differences between significant DMRs and the background dataset. Conversely, the intron regions show a higher fraction of total length in the background dataset compared to significant DMRs. Examination of chart 2305 reveals that the inter regions have a higher representation in the background dataset, whereas the island region shows significant enrichment in significant DMRs compared to the background.

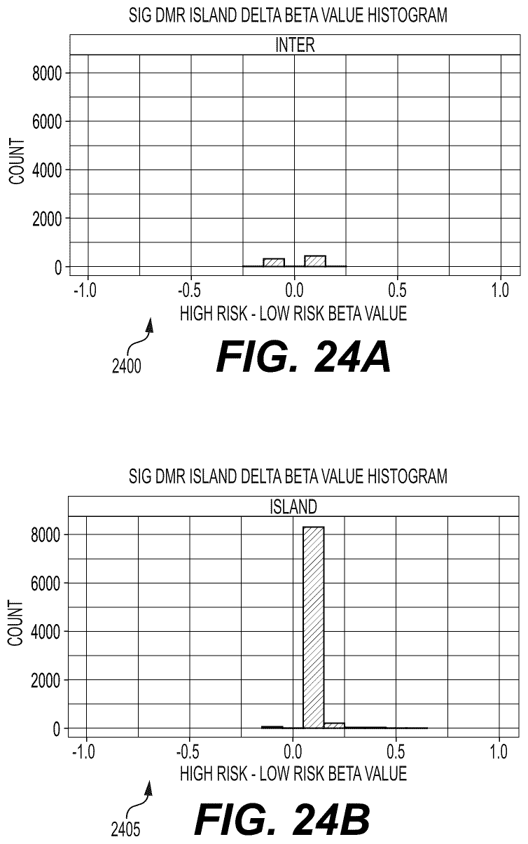

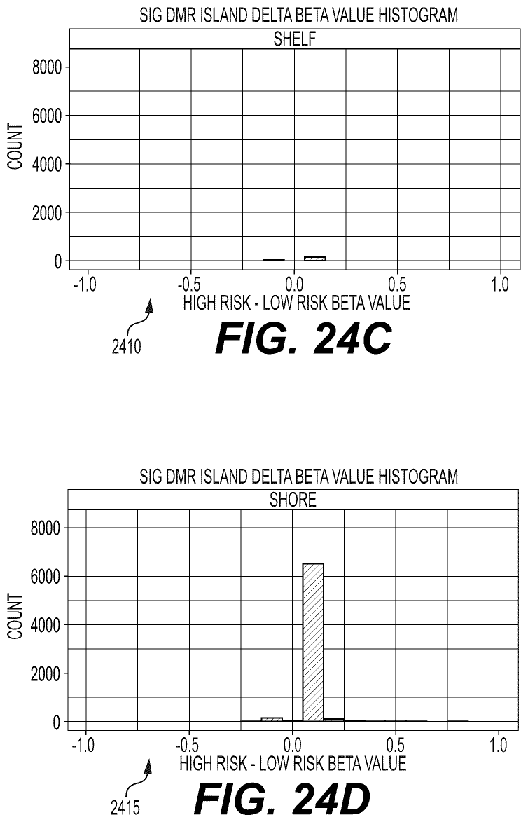

FIGS. 24A-24D present histograms 2400-2415, respectively, that depict the delta beta values for significant DMRs related to CpG features. The x-axis of each represents the difference in methylation levels between high-risk and low-risk groups. The y-axis in each represents the count of DMRs. Inter histogram 2400 shows a small count of DMRs with the beta values being close to zero, indicating minimal differences in methylation levels between high-risk and low-risk groups for inter regions. Island histogram 2405 shows a high count of DMRs with most beta values close to zero but exhibiting a spread towards hypermethylation, indicating a higher number of regions with increased methylation in high-risk subjects. Shelf histogram 2410 shows a small count of DMRs with the beta values being close to zero, indicating minimal differences in methylation levels for shelf regions. Shore histogram 2415 shows a significant count of DMRs with a notable trend towards hypermethylation, suggesting that shore regions have a higher number of regions with increased methylation in high-risk subjects.

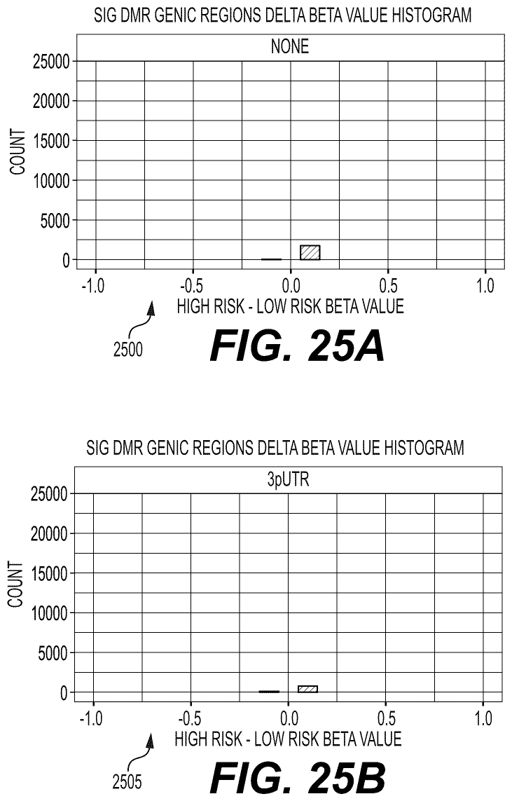

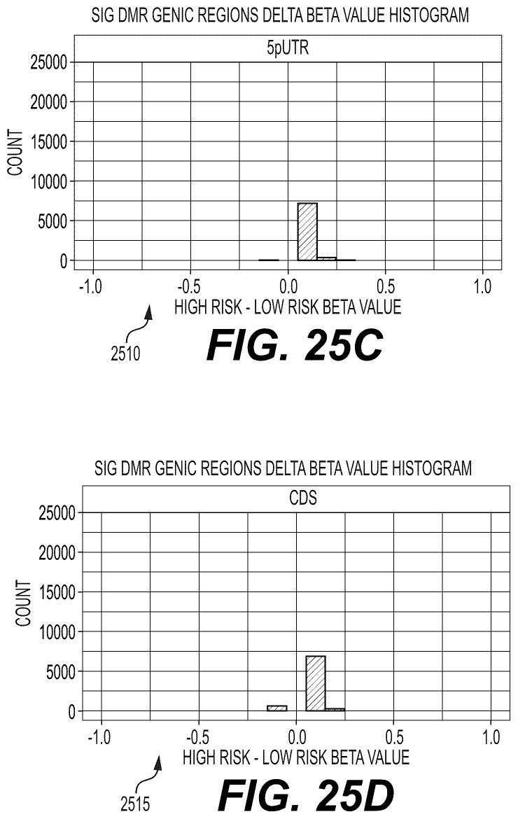

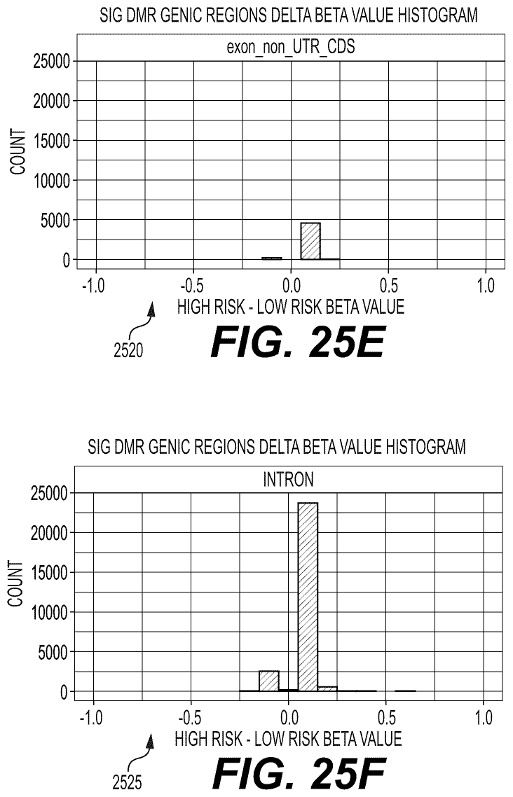

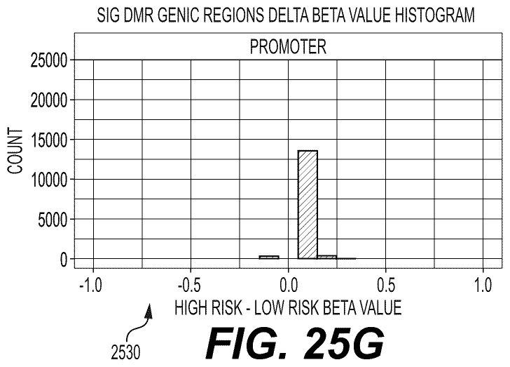

FIGS. 25A-25G present histograms 2500-2530, respectively, that depict the beta values for significant DMRs across various genic regions. The x-axis in each represents the difference in methylation levels between high-risk and low-risk groups. The y-axis in each represents the count of DMRs. None histogram 2500 shows a small count of DMRs and the delta beta values are close to zero, indicating minimal differences in methylation levels between high-risk and low-risk groups. 3pUTR histogram 2505 shows a small count of DMRS and the delta beta values are close to zero, indicating minimal differences in methylation levels between high-risk and low-risk groups. 5pUTR histogram 2510 shows a small count of DMRs but displays a notable peak close to zero with a spread towards hypermethylation, indicating some regions with increased methylation in high-risk groups. CDS histogram 2515 shows a small count of DMRs with most beta values close to zero, indicating minimal differences in methylation levels between high-risk and low-risk groups. Exon_non_UTR_CDS histogram 2520 shows a small count of DMRs with most beta values close to zero, indicating minimal differences in methylation levels between high-risk and low-risk groups. Intron histogram 2525 shows a high count for DMRs with most beta values close to zero but also exhibiting a higher prevalence of regions with hypermethylation in high-risk groups. Promoter histogram 2530 shows a significant count of DMRs and a notable peak close to zero with a spread towards hypermethylation, indicating that promotor regions have a higher number of regions with increased methylation in high-risk groups.

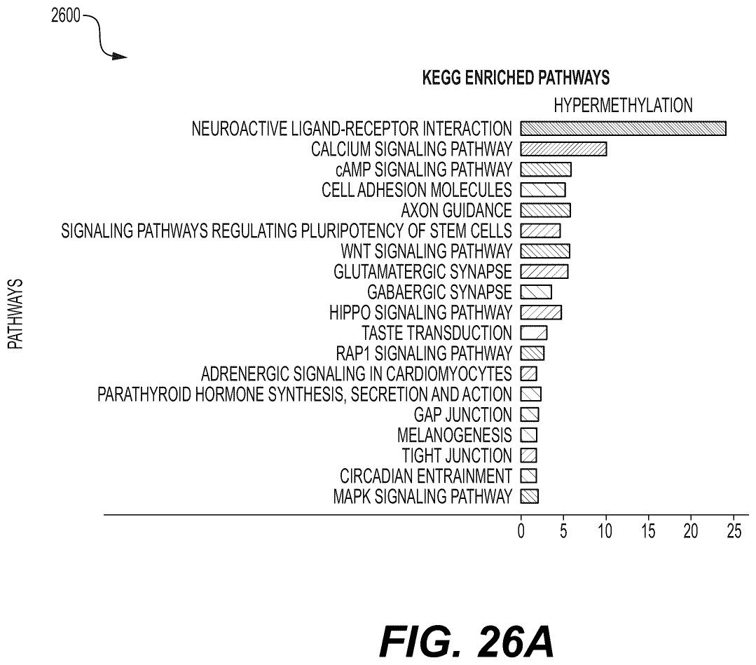

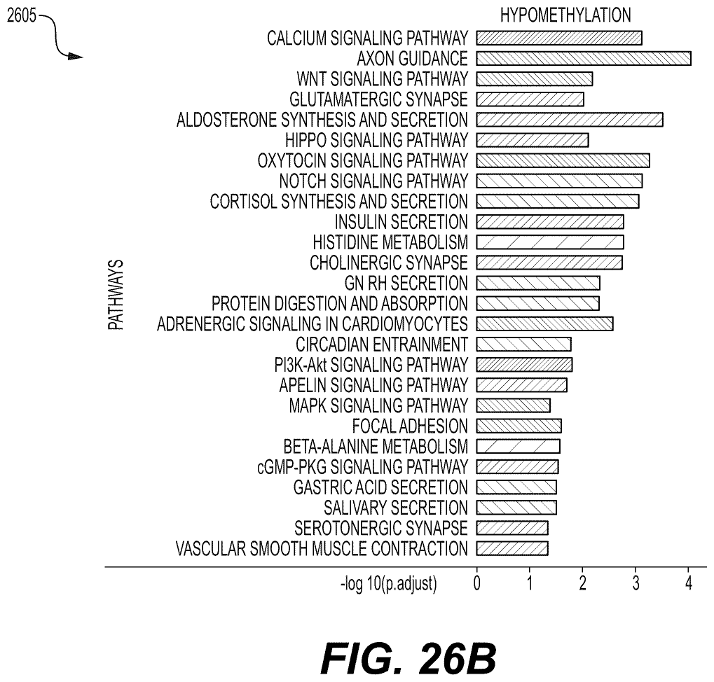

FIGS. 26A and 26B present panels 2600 and 2605, respectively, that collectively illustrate KEGG enrichment analysis for DMRs in high-risk versus low-risk participant groups. For each panel, the x-axis represents the −log 10 of the adjusted p-value for the enrichment of each pathway, whereas the y-axis lists the KEGG pathways that are significantly enriched for the panel type. Panel 2600 lists pathways enriched for hypermethylated genes in the high-risk group. The most significantly enriched pathways, neuroactive ligand-receptor interaction, indicates a potential disruption in neurotransmission. Panel 2605 lists pathways enriched for hypomethylated genes in the high-risk group. The most significantly enriched pathways, e.g., calcium signaling pathway and axon guidance, indicates complex regulatory changes in high-risk groups.

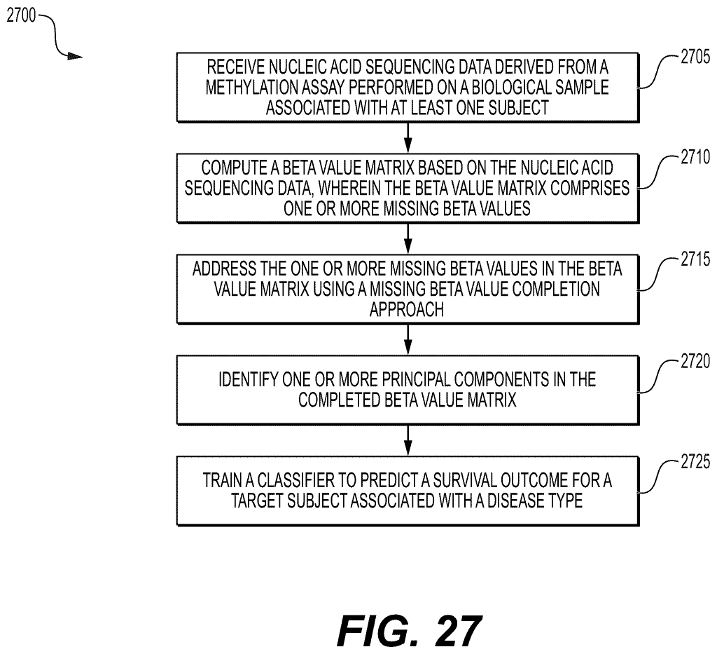

Referring now to FIG. 27, an exemplary workflow 2700 is provided for training a prognostic classifier configured to predict a survival outcome for a target subject associated with a disease type. Aspects of the exemplary workflow 2700 may be performed in accordance with some or all components described in FIGS. 1A and 1B.

At step 2705, system 100 may receive DNA sequencing data derived from a methylation assay performed on a biological sample associated with at least one subject. In an aspect, DNA sequencing data may be collected from biological samples, such as blood samples, obtained from subjects diagnosed with various hematologic malignancies. These samples may undergo a methylation assay, which measures the DNA methylation levels across specific regions of the genome. Such methylation assays are discussed further in reference to FIG. 2A, step 210.

At step 2710, system 100 may compute a beta value matrix based on the DNA sequencing data, as described in reference to step 215 in FIG. 2A. In an aspect, the DNA sequencing data derived at step 2705 may then be processed to compute a beta value matrix, where each value in the matrix represents the methylation level of a specific cytosine in a given genomic context. Beta values range from 0 (complete unmethylation) to 1 (complete methylation). In an aspect, the beta value matrix may contain missing values due to various factors, such as insufficient sequencing coverage or technical issues during data acquisition. Addressing these missing values may promote the integrity and accuracy of the subsequent analysis.

At step 2715, system 100 may address the missing beta values in the beta value matrix using a missing beta value completion approach. In an aspect, two common completion approaches include a non-imputation approach, where missing values are ignored, and an imputation approach, where missing values are filled using mean or median imputation based on the available training data, as described in reference to FIG. 2A, step 220. If leveraging the latter approach, before imputation, regions with more than a predetermined percentage of missing values (e.g., 20%) may be removed to maintain data quality. This step is performed so that the dataset remains as complete and informative as possible, promoting the robustness of the predictive model.

At step 2720, system 100 may identify one or more PCs in the completed beta value matrix. In an aspect, once the missing values are addressed (e.g., by either ignoring the missing values using the non-imputation approach or by filling in the missing values using the median imputation approach), the completed beta value matrix may still contain a vast number of features, leading to potential overfitting and computational inefficiencies. To mitigate this, a dimensionality reduction technique, such as PCA, is applied. PCA transforms the high-dimensional space into a smaller set of uncorrelated PCs that capture the most significant variance in the data, as described further above. This reduction effectively summarizes the essential information while discarding noise and redundancies, thereby improving the model's predictive accuracy and efficiency.

At step 2725, system 100 may train a classifier to predict a survival outcome for a target subject associated with a disease type. In an aspect, the PCs identified in step 2720 may be utilized to train a classifier designed to predict survival outcomes for subjects with hematologic malignancies, such as one or more of those described herein. An RSF model may be employed for this purpose. The RSF model constructs multiple decision trees using bootstrapped samples from the training data, with each tree providing a survival function. The aggregated survival predictions from all trees yield the final survival prediction for each subject. The model may be validated using techniques such as cross-validation and hyperparameter tuning to ensure robust and accurate survival predictions, as described above. In an aspect, the trained classifier may stratify subjects into different risk groups, aiding clinicians in making informed treatment decisions and improving subject outcomes.

In general, any process discussed in this disclosure that is understood to be computer-implementable may be performed by one or more processors of a computer system, such as system environment 110, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer server. A processor may be a central processing unit (CPU), a graphics processing unit (GPU), or any suitable types of processing unit.

A computer system, such as system environment 110, may include one or more computing devices. If the one or more processors of the computer system are implemented as a plurality of processors, the plurality of processors may be included in a single computing device or distributed among a plurality of computing devices. If a system environment comprises a plurality of computing devices, the memory of the computer system may include the respective memory of each computing device of the plurality of computing devices.

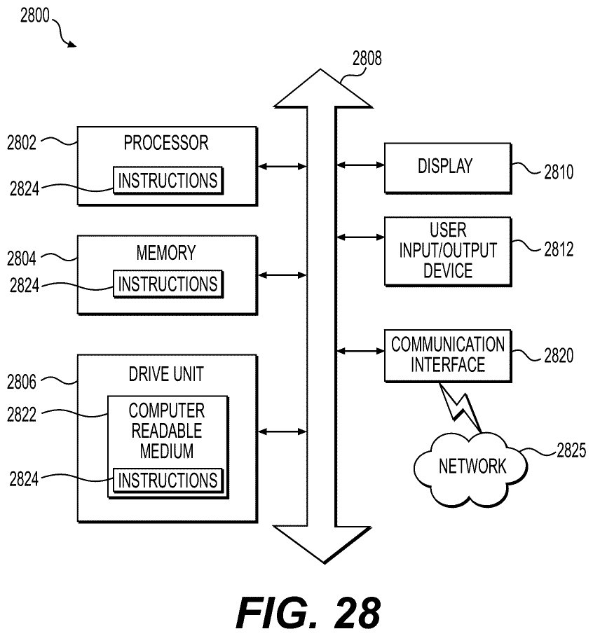

FIG. 28 is a simplified functional block diagram of a computer system 2800 that may be configured as a computing device for executing the processes described herein, according to exemplary embodiments of the present disclosure. In various embodiments, any of the systems herein may be an assembly of hardware including, for example, a data communication interface 2820 for packet data communication. The platform also may include a central processing unit (“CPU”) 2802, in the form of one or more processors, for executing program instructions. The platform may include an internal communication bus 2808, and a storage unit 2806 (such as ROM, HDD, SDD, etc.) that may store data on a computer readable medium 2822, although the system 2800 may receive programming and data via network communications via electronic network 2825 (e.g., voice, video, audio, images, or any other data over the electronic network 2825). The system 2800 may also have a memory 2804 (such as RAM) storing instructions 2824 for executing techniques presented herein, although the instructions 2824 may be stored temporarily or permanently within other modules of system 2800 (e.g., processor 2802 and/or computer readable medium 2822). The system 2800 also may include input and output ports 2812 and/or a display 2810 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as “about,” “approximately,” “substantially,” and “generally,” are used to indicate a possible variation of ±10% of a stated or understood value. In addition, the term “between” used in describing ranges of values is intended to include the minimum and maximum values described herein. The use of the term “or” in the claims and specification is used to mean “and/or” unless explicitly indicated to refer to alternatives only if the alternatives are mutually exclusive, although the disclosure supports a definition that refers to only alternatives and “and/or.” As used herein “another” may mean at least a second or more.

As used herein, the term “user” generally encompasses any person or entity, such as a researcher and/or a care provider (e.g., a doctor, etc.), that may desire information, resolution of an issue, or engage in any other type of interaction with a provider of the systems and methods described herein (e.g., via an application interface resident on their electronic device, etc.). The term “electronic application” or “application” may be used interchangeably with other terms like “program,” or the like, and generally encompasses software that is configured to interact with, modify, override, supplement, or operate in conjunction with other software.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method, the computer-implemented method comprising:
receiving, at a computer system, nucleic acid sequencing data derived from a methylation assay performed on a biological sample associated with at least one subject;
computing, using a processor associated with the computer system, a beta value matrix based on the nucleic acid sequencing data, wherein the beta value matrix comprises one or more missing beta values;
addressing, using the processor, the one or more missing beta values in the beta value matrix using a missing beta value completion approach;
identifying, using the processor, one or more principal components in the completed beta value matrix; and
training, using the one or more principal components in combination with a predetermined set of clinical variables, a classifier to predict a survival outcome for a target subject associated with a disease type.

2. The computer-implemented method of claim 1, wherein the methylation assay is a cell-free DNA (cfDNA) targeted methylation assay and the biological sample is one of: a blood plasma sample or a blood serum sample.

3. The computer-implemented method of claim 1, wherein each beta value in the beta value matrix ranges between 0 to 1.

4. The computer-implemented method of claim 1, wherein the addressing the one or more missing beta values using the missing beta value completion approach comprises addressing via one of: a non-imputation approach or an imputation approach.

5. The computer-implemented method of claim 4, wherein the addressing the one or more missing beta values using the non-imputation approach comprises ignoring the one or more missing beta values.

6. The computer-implemented method of claim 4, wherein the addressing the one or more missing beta values using the imputation approach comprises:
constructing filtered nucleic acid sequencing data by removing regions in the nucleic acid sequencing data containing greater than a threshold percentage of missing beta values;
calculating one or more median imputation values from the constructed filtered nucleic acid sequencing data; and
filling in the one or more missing beta values with the calculated one or more median imputation values.

7. The method of claim 1, wherein the classifier is a random survival forest (RSF) classifier.

8. The method of claim 1, wherein the predetermined set of clinical variables include one or more of: heme subtype, race, age, sex, highest clinical stage, body-mass index (BMI), smoking status, and drinking status.

9. The method of claim 1, wherein the training the classifier to predict the survival outcome comprises configuring the classifier to stratify the target subject into at least a high-risk and a low-risk group.

10. The method of claim 9, further comprising configuring the classifier to stratify the target subject into a medium-risk group.

11. The method of claim 1, wherein the disease type is a hematologic malignancy.

12. The method of claim 11, wherein the hematologic malignancy is at least one of: B-cell lymphoma, chronic lymphocytic leukemia small lymphocytic lymphoma (CLL_SLL), diffuse large B-cell lymphoma (DLBCL), essential thrombocythemia, follicular lymphoma, Hodgkin lymphoma, lymphoplasmacytic, mucosa-associated lymphoid tissue nodal marginal zone lymphoma (MALT NMZL), mantle cell, myelodysplastic syndrome (MDS), monoclonal gammopathy of undetermined significance (MGUS), plasma cell myeloma, plasma cell neoplasm, and polycythemia vera.

13. A system, comprising:
one or more processors;
one or more computer readable media storing instructions that are executable by the one or more processors to perform operations to:
receive nucleic acid sequencing data derived from a methylation assay performed on a biological sample associated with at least one subject;
compute a beta value matrix based on the nucleic acid sequencing data, wherein the beta value matrix comprises one or more missing beta values;
address the one or more missing beta values in the beta value matrix using a missing beta value completion approach;
identify one or more principal components in the completed beta value matrix; and
train, using the one or more principal components in combination with a predetermined set of clinical variables, a classifier to predict a survival outcome for a target subject associated with a disease type.

14. The system of claim 13, wherein the methylation assay is a cell-free DNA (cfDNA) targeted methylation assay and the biological sample is one of: a blood plasma sample or a blood serum sample.

15. The system of claim 13, wherein the operations to address the one or more missing beta values using the missing beta value completion approach comprise operations to address via one of: a non-imputation approach or an imputation approach.

16. The system of claim 15, wherein the operations to address the one or more missing beta values using the non-imputation approach comprise operations to ignore the one or more missing beta values.

17. The system of claim 15, wherein the operations to address the one or more missing beta values using the imputation approach comprise operations to:
construct filtered nucleic acid sequencing data by removing regions in the nucleic acid sequencing data containing greater than a threshold percentage of missing beta values;
calculate one or more median imputation values from the constructed filtered nucleic acid sequencing data; and
fill in the one or more missing beta values with the calculated one or more median imputation values.

18. The system of claim 13, wherein the classifier is a random survival forest (RSF) classifier.

19. The system of claim 13, wherein the predetermined set of clinical variables include one or more of: heme subtype, race, age, sex, highest clinical stage, body-mass index (BMI), smoking status, and drinking status.

20. The system of claim 19, wherein the operations to train the classifier to predict the survival outcome comprise operations to: configure the classifier to stratify the target subject into at least one of a high-risk or a low-risk group.

21. The system of claim 20, wherein the operations further comprise configuring the classifier to stratify the target subject into a medium-risk group.

22. The system of claim 13, wherein the disease type is a hematologic malignancy.

23. The system of claim 22, wherein the hematologic malignancy is at least one of: B-cell lymphoma, chronic lymphocytic leukemia small lymphocytic lymphoma (CLL_SLL), diffuse large B-cell lymphoma (DLBCL), essential thrombocythemia, follicular lymphoma, Hodgkin lymphoma, lymphoplasmacytic, mucosa-associated lymphoid tissue nodal marginal zone lymphoma (MALT NMZL), mantle cell, myelodysplastic syndrome (MDS), monoclonal gammopathy of undetermined significance (MGUS), plasma cell myeloma, plasma cell neoplasm, and polycythemia vera.

24. A non-transitory computer-readable medium storing computer-executable instructions which, when executed by a system, cause the system to perform operations comprising:
receiving, at a computer system, nucleic acid sequencing data derived from a methylation assay performed on a biological sample associated with at least one subject;
computing, using a processor associated with the computer system, a beta value matrix based on the nucleic acid sequencing data, wherein the beta value matrix comprises one or more missing beta values;
addressing, using the processor, the one or more missing beta values in the beta value matrix using a missing beta value completion approach;
identifying, using the processor, one or more principal components in the completed beta value matrix; and
training, using the one or more principal components in combination with a predetermined set of clinical variables, a classifier to predict a survival outcome for a target subject associated with a disease type.
